(19) United States
(12) Patent Application Publication
(10) Pub. No.: US 2014/0037682 A1
(43) Pub. Date: Feb. 6, 2014

(54) HIV/SIV VACCINES FOR THE GENERATION OF MUCOSAL AND SYSTEMIC IMMUNITY

(71) Applicant: UNIVERSITY OF MIAMI, Miami, FL (US)

(72) Inventors: ECKHARD R. PODACK, COCONUT GROVE, FL (US); NATASA STRBO, MIAMI BEACH, FL (US)

(73) Assignee: UNIVERSITY OF MIAMI, Miami, FL (US)

(21) Appl. No.: 14/052,309

(22) Filed: Oct. 11, 2013

Related U.S. Application Data
(62) Division of application No. 13/129,920, filed on May 18, 2011.
(60) Provisional application No. 61/116,971, filed on Nov. 21, 2008.

(51) Int. Cl. A61K 39/21

(52) U.S. Cl. CPC A61K 39/21
USPC 424/208.1

(57) ABSTRACT

Compositions of genetically engineered, secreted gp96 (gp69-Ig) induced strong mucosal and systemic immune responses and CD8 expansion that was independent of CD4 help. Immunization of patients with gp96-Ig immunization is especially attractive for induction of mucosal and systemic immunity to SIV/HIV and other diseases.

### CROSS-REFERENCE TO RELATED APPLICATIONS

The present application claims the priority of U.S. provisional patent application No. 61/116,971 filed Nov. 21, 2008, which is incorporated herein by reference in its entirety.

### STATEMENT REGARDING FEDERALLY SPONSORED RESEARCH OR DEVELOPMENT

The United States Government may have certain rights in this invention pursuant to the National Institute of Allergy and Infectious Diseases Grant No. AI073234.

### FIELD OF THE INVENTION

Embodiments of this invention relates to T cell based vaccines or compositions which generate mucosal and systemic and systemic immunity to pathogens, including, HIV and SIV. In particular, the vaccine elicits powerful mucosal and systemic effector CD8 T cell response that combats the pathogen in the mucosa before the establishment of a systemic infection.

### BACKGROUND

Citation or discussion of a reference herein shall not be construed as an admission that such is prior art to the present invention.

Vaccination has eradicated certain diseases such as polio, tetanus, chicken pox, and measles in many countries. This approach has exploited the ability of the immune system to resist and prevent infectious diseases. Traditional ways of preparing vaccines include the use of inactivated or attenuated pathogens. A suitable inactivation of the pathogenic microorganism renders it harmless as a biological agent but does not destroy its immunogenicity. Injection of these “killed” particles into a host will then elicit an immune response capable of preventing a future infection with a live microorganism. However, a major concern in the use of inactivated pathogens as vaccines is the failure to inactivate all the microorganisms. Even when this is accomplished, since killed pathogens do not multiply in their host, or for other unknown reasons, the immunity achieved is often incomplete, short lived and requires multiple immunizations. Finally, the inactivation process may alter the microorganism's antigens, rendering them less effective as immunogens.

Attenuation refers to the production of strains of pathogenic microorganisms which have essentially lost their disease-producing ability. One way to accomplish this is to subject the microorganism to unusual growth conditions and/or frequent passage in cell culture. Mutants are then selected which have lost virulence but yet are capable of eliciting an immune response. Attenuated pathogens often make good immunogens as they actually replicate in the host cell and elicit long lasting immunity. However, several problems are encountered with the use of live vaccines, the most worrisome being insufficient attenuation and the risk of reversion to virulence.

An alternative to the above methods is the use of subunit vaccines. This involves immunization only with those components which contain the relevant immunological material. A new promising alternative is the use of DNA or RNA as vaccines. Vaccines are often formulated and inoculated with various adjuvants. The adjuvants aid in attaining a more durable and higher level of immunity using small amounts of antigen or fewer doses than if the immunogen were administered alone. The mechanism of adjuvant action is unpredictable, complex and not completely understood.

An organism's immune system reacts with two types of responses to pathogens or other harmful agents—humoral response and cell-mediated response. When resting B cells are activated by antigen to proliferate and mature into antibody-secreting cells, they produce and secrete antibodies with a unique antigen-binding site. This antibody-secreting reaction is known as the humoral response. On the other hand, the diverse responses of T cells are collectively called cell-mediated immune reactions. There are two main classes of T cells—cytotoxic T cells and helper T cells. Cytotoxic T cells directly kill cells that are infected with a virus or some other intracellular microorganism. Helper T cells, by contrast, help stimulate the responses of other cells: they help activate macrophages, dendritic cells and B cells. Both cytotoxic T cells and helper T cells recognize antigen in the form of peptide fragments that are generated by the degradation of foreign protein antigens inside the target cell, and both, therefore, depend on major histocompatibility complex (MHC) molecules, which bind these peptide fragments, carry them to the cell surface, and present them there to the T cells. MHC molecules are typically found in abundance on antigen-presenting cells (APCs).

Antigen-presenting cells (APCs), such as macrophages and dendritic cells, are key components of innate and adaptive immune responses. Antigens are generally ‘presented’ to T cells or B cells on the surfaces of other cells, the APCs. APCs can trap lymph- and blood-borne antigens and, after internalization and degradation, present antigenic peptide fragments, bound to cell-surface molecules of the major histocompatibility complex (MHC), to T cells. APCs may then activate T cells (cell-mediated response) to clonal expansion, and these daughter cells may either develop into cytotoxic T cells or helper T cells, which in turn activate B (humoral response) cells with the same MHC-bound antigen to clonal expansion and specific antibody production.

Two types of antigen-processing mechanisms have been recognized. The first type involves uptake of proteins through endocytosis by APCs, antigen fragmentation within vesicles, association with class II MHC molecules and expression on the cell surface. This complex is recognized by helper T cells expressing CD4. The other is employed for proteins, such as viral antigens, that are synthesized within the cell and appears to involve protein fragmentation in the cytoplasm. Peptides produced in this manner become associated with class I MHC molecules and are recognized by cytotoxic T cells expressing CD8. Stimulation of T cells involves a number of accessory molecules expressed by both T cell and APC. Co-stimulatory molecules are those accessory molecules that promote the growth and activation of the T cell. Upon stimulation, co-stimulatory molecules induce release of cytokines, such as interleukin 1 (IL-1) or interleukin 2 (IL-2), interferon, etc., which promote T cell growth and expression of surface receptors.

### SUMMARY

This Summary is provided to present a summary of the invention to briefly indicate the nature and substance of the invention. It is submitted with the understanding that it will not be used to interpret or limit the scope or meaning of the claims.

In a preferred embodiment, a composition comprises an isolated nucleic acid encoding at least one molecule comprising: at least one heat shock protein, at least one immunogen, fragments, variants, mutants, derivatives or combinations thereof. Preferably, a heat shock protein comprises at least one of hsp70, hsp90, gp96, fragments, mutants, derivatives, variants, or combinations thereof. Preferably, an immunogen comprises at least one of; bacterial antigen, viral antigen, parasitic antigen, prion, tumor antigen, or combinations thereof.

The isolated nucleic acid of claim 21, wherein the encoded molecule when administered to a cell is secreted, membrane bound, retained endogenously or combinations thereof.

In another preferred embodiment, a method of inducing HIV/SIV antigen specific mucosal and systemic immunity in vivo, comprising administering to a patient in need thereof; a therapeutically effective amount of an antigenic composition comprising gp96. Preferably, the HIV/SIV antigen specific mucosal and systemic immunity comprises induction of an antigen specific T cell immune response.

In another preferred embodiment, the antigen specific T cell response is polyspecific comprising CD8 and CD4 T cells.

In another preferred embodiment, the HIV/SIV antigen specific mucosal and systemic immunity further comprises innate dendritic cell, natural killer cells (NK), and memory CD8⁺ T cells.

In another preferred embodiment, the antigenic composition comprising: an isolated cell having a plasmid encoding gp96-Ig, HIV/SIV antigens retanef (Rev-Tat-Nef), gag, gp160, fragments, variants, mutants, derivatives or combinations thereof. Preferably, the retanef comprises at least one of Rev, Tat, Nef; fragments, variants, mutants, derivatives or combinations thereof.

In another preferred embodiment, the isolated cell expresses endogenous, membrane bound, secreted or combinations thereof of at least one of the molecules comprising: gp96, retanef, gag, gp160, fragments, variants, mutants, derivatives or combinations thereof. Preferably, the cell comprises autologous, syngeneic, heterologous, xenogeneic cells, cell lines, or combinations thereof.

In another preferred embodiment, a method of preventing HIV in a patient at risk of being infected with HIV, or treating a patient, infected with HIV, comprising: administering to the patient in need thereof; a therapeutically effective amount of antigen comprising gp96.

In another preferred embodiment, the antigen comprising gp96 induces an HIV/SIV antigen specific mucosal and systemic immunity comprising an antigen specific T cell immune response, wherein the T cell response is polyspecific comprising CD8⁺ and CD4⁺ T cells.

In another preferred embodiment, the HIV/SIV antigen specific mucosal and systemic immunity further comprises innate dendritic cell, natural killer cells (NK), CD103⁺ cells, CD8⁺ CD 103⁺ T cells, and/or memory CD8⁺ T cells.

In another preferred embodiment, the HIV/SIV antigen induces immune cells comprising central memory T cells (T_{CM}; CD95⁺ CD28⁺), effector memory T cells (T_{EM}; CD95⁺ CD28⁻) or naive T cells (CD95^{low} CD28^{int}).

In another preferred embodiment, the HIV/SIV antigen comprising: an isolated cell having a plasmid encoding gp96-Ig, HIV/SIV antigens retanef (Rev-Tat-Nef), gag, gp160, fragments, variants, mutants, derivatives or combinations thereof.

In another preferred embodiment, the retanef comprises at least one of Rev, Tat, Nef, fragments, variants, mutants, derivatives or combinations thereof.

In another preferred embodiment, the isolated cell expresses membrane bound and/or secreted molecules of gp96, retanef, gag, gp160, fragments, variants, mutants, derivatives or combinations thereof.

In another preferred embodiment, an isolated nucleic acid encoding at least one molecule comprising: gp96-Ig, HIV/SIV antigens retanef (Rev-Tat-Nef), gag, gp160, fragments, variants, mutants, derivatives or combinations thereof.

In another preferred embodiment, the encoded molecules are endogenous, membrane bound, secreted or combinations thereof.

In another preferred embodiment, a fusion protein comprising at least one of: gp96-Ig, HIV/SIV antigens retanef (Rev-Tat-Nef), gag, gp160, fragments, variants, mutants, derivatives or combinations thereof.

In another preferred embodiment, a fusion protein comprising at least one of: gp96-Ig, immunogens, fragments, variants, mutants, derivatives or combinations thereof.

In another preferred embodiment, an isolated cell comprising a nucleic acid molecule encoding at least one or more of: gp96-Ig, HIV/SIV antigens retanef (Rev-Tat-Nef), gag, gp160, fragments, variants, mutants, derivatives or combinations thereof.

In another preferred embodiment, an isolated cell comprising a nucleic acid molecule encoding at least one or more of: gp96-Ig, immunogens, fragments, variants, mutants, derivatives or combinations thereof; wherein at least one immunogen is secreted.

In another preferred embodiment, a method of inducing a Human Immunodeficiency Virus (HIV) specific immune response in vivo or in vitro, comprising: administering to the patient in need thereof, a therapeutically effective amount of antigen comprising gp96.

In another preferred embodiment, the antigen comprising gp96 induces an HIV/SIV antigen specific mucosal and systemic immunity comprising an antigen specific T cell immune response. In another preferred embodiment, the antigen specific T cell response is polyspecific comprising CD8⁺ and CD4⁺ T cells. Preferably, the antigen specific T cells co-express and produce IFNγ and IL-2.

In another preferred embodiment, the HIV/SIV antigen specific mucosal and systemic immunity further comprises innate dendritic cell, natural killer cells (NK), CD103⁺ cells, CD8⁺ CD103⁺ T cells, and/or memory CD8⁺ T cells.

In another preferred embodiment, the HIV/SIV antigen induces immune cells comprising central memory T cells (T_{CM}; CD95⁺ CD28⁺), effector memory T cells (T_{EM}; CD95⁺ CD28⁻) or naive T cells (CD95^{low} CD28^{int}.).

In another preferred embodiment, the HIV/SIV antigen comprising: an isolated cell having a plasmid encoding gp96-Ig, HIV/SIV antigens retanef (Rev-Tat-Nef), gag, gp160, fragments, variants, mutants, derivatives or combinations thereof.

In another preferred embodiment, a method of inducing an antigen specific mucosal and systemic immune response, comprising; administering to a patient in need thereof, a composition comprising at least one molecule comprising: a heat shock protein, at least one immunogen, fragments, variants, mutants, derivatives or combinations thereof.

In another preferred embodiment, a heat shock protein comprises at least one of hsp70, hsp90, gp96, fragments, mutants, derivatives, variants, or combinations thereof.

In another preferred embodiment, an immunogen comprises at least one of bacterial antigen, viral antigen, parasitic antigen, tumor antigen, or combinations thereof. In another preferred embodiment, the molecule is administered as a cell secreted molecule, wherein a patient is contacted with a cell comprising a nucleic acid encoding the molecule wherein the molecule is secreted by the cell. In another preferred embodiment, the cell comprises autologous, syngeneic, heterologous, xenogeneic cells, cell lines, or combinations thereof.

Other aspects are described infra.

### BRIEF DESCRIPTION OF THE DRAWINGS

FIGS. 1A-1C show the expression of gp96-Ig and SIV antigens in 293 SIV-vaccine cells. Study design and immunization regiment (FIG. 1A): 293 cells were transfected with B45-gp96-Ig-rtn and plasmids pCMV-SIV-gp160 and pCMV-SIV-gag. Cells were selected with G418 and checked for expression of gp96-Ig, net gag and env by Western blots. Lane 1: 293. Lane 2: 293-gp96-ReTaNef-gp96-Ig/SIVgag/SIVgp 160. Lane 3: 293-SIVReTaNef-gp96-Ig/SIVgag/SIVgp160 irradiated cells. FIG. 1B: 1×10⁶ cells were plated in 1 ml for 24 h and gp96-Ig production was determined by ELISA using anti-human IgG antibody for detection and human IgG as a standard. FIG. 1C: Eight naïve female Rhesus macaques, that carry the MHC class I Mamu-A*01 molecule were enrolled in the study and divided in four groups. Schedule of immunization shown in black arrows. Macaques in groups I, II and III were immunized at weeks 0, 4 and 25 with intraperitoneal inoculations of irradiated vaccine cells (293-gp96-Ig-SIV) that secrete 1, 5 or 50 mg of gp96-Ig at the rate of 106 cells/24 h. Irradiated 293 cells (50×10⁶) were injected in the animals from group IV (control). Short arrows (5 days after each vaccination) represent time of the collection of PBL, lymph nodes, BAL and rectal and vaginal pinches biopsies.

FIGS. 2A-2C show that gp96-SIV vaccination increases SIV-specific CD8 T cells in the blood. Eight Rhesus macaques were sorted into 4 groups and immunized with SIV-gp96-vaccine cells by the intra-peritoneal route. The number of cells injected secreting the quantity of product as indicated within 24 h (by ELISA). Vaccine cells were administered 3 times at 0, 4 and 25 weeks (black arrows), and animal M964 received 293 alone (control). 5 days after every immunization, blood was collected, and PBMC obtained by density-gradient centrifugation. FIG. 2A: SIV-Gag- and Tat-specific CD8 T cells were detected by Mamu-A*01/Gag181-189 CM9 (CTPYDINQM; Gag CM9) and Tat 28-35 SL8 (TTPESANL; Tat SL8) tetramer staining. In addition, cells were stained with monoclonal antibody to CD8β and analyzed by flow cytometry. After gating on the CD8β⁺ population, the percentage of tetramer-positive cells was determined at each time point (vaccine induces SIV-gag- and tat-specific CD8 T cells in the peripheral blood). FIG. 2B: Frequency of gag- tat- and env-specific cells in PBMC of vaccinated macaques as measured by IFN g-ELISPOT assay following the gp96-Ig-SIV vaccination. The y-axis represents SFC, Spot-forming cells per 2×10⁵ cells; and x-axis time (weeks). PBMC were incubated on ELISPOT plates in the presence of peptides pools of 15-meric peptides overlapping by 11 amino acids covering the entire gag, tat and env proteins of SIVmac251. Controls include animal M694 that received only 293 cells. FIG. 2C: Representative FACS plots for monkey M943 (group II) showing expression of CD28 and CD95 on gated CD8 T cells (gray contour plot) and Gag CM9⁺ CD8 T cells (red contour plot) in the blood. Numbers on the FACS plots represent frequency of central memory (CM) and effector memory (EM)-positive cells as a percent of total Gag CM9⁺ cells as defined by the expression of CD28 and CD95 (TCM CD28⁺ CD95⁺ and TEM CD28⁻ CD95⁺).

FIGS. 3A-3B show the SIV-specific immune responses in secondary lymphoid organs. FIG. 3A: SIV-Gag- and Tat-specific CD8 T cells were detected by Mamu-A*01/Gag181-189 CM9 (CTPYDINQM; Gag CM9) and Tat 28-35 SL8 (TTPESANL; Tat SL8) tetramer staining. In addition, cells were stained with monoclonal antibody to CD8β and analyzed by floe cytometry. After gating on the CD8β⁺ population, the percentage of tetramer positive cells was determined at each time point (vaccine induces SIV-gag- and tat-specific CD8 T cells in the peripheral blood). FIG. 3B shows representative FACS plots for monkey M620 (group I), M943 (group II) and M947 (group II) showing expression of CD28 and CD95 on gated CD8 T cells (gray contour plot) and gag CM9⁺ CD8 T cells (red contour plot) in the inguinal lymph nodes at week 1 and week 26. Numbers on the FACS plots represent the frequency of central memory (TCM) and effector memory (TEM) positive cells as a percent of total tetramer positive cells as defined by the expression of CD28 and CD95 (TCM CD28⁺CD95⁺ and TEM CD28⁻CD95⁺).

FIGS. 4A-4C show SIV-gp96 immunization-induced SIV-Gag- and Tat-specific CD8 T cells in the rectal mucosa lamina propria. Eight Rhesus macaques were sorted into 4 groups and immunized with gp96-SIV by the intra-peritoneal route. The number of cells injected secreting the quantity of product as indicated within 24 h (by ELISA). Vaccine cells were administered 3 times at 0, 4 and 25 weeks, and two animals received 293 alone (control). 5 days after every immunization, samples were harvested from the rectal mucosa. FIG. 4A: SIV-Gag- and Tat-specific CD8 T cells were detected by Mamu-A*01/Gag181-189 CM9 (CTPYDINQM; Gag_CM9) and Tat 28-35 SL8 (TTPESANL; Tat SL8) tetramer staining. In addition, cells were stained with monoclonal antibody to CD8β and analyzed by flow cytometry. After gating on the CD8β⁺ population, the percentage of tetramer-positive cells was determined at each time point. The individual data for each monkey has been plotted. FIG. 4B: Dot plots from representative monkey (M944 that has been vaccinated with 5 μg of Gp96-Ig-SIV) demonstrating binding of Gag CM9 tetramer on CD8 T cells from rectal mucosa lamina propria. Numbers represent percent of tetramer+ cells within CD8 gate. FIG. 4C: Frequency of Gag CM9 tetramer⁺ (right) and Tat SL8 tetramer⁺ (left) CD8 T cells in rectal lamina propria and inguinal lymph nodes from monkeys that received 5 μg of Gp96-Ig-SIV (Group II) and control monkey (Group IV) at week 26. Bars represent mean±SE of the percentage tetramer⁺ cells (n=2) ***p<0.001 (compared with inguinal lymph node).

FIGS. 5A, 5B show that Gp96-Ig-SIV vaccine induces Gag- and Tat-specific CD8 T cells in the lamina propria from jejunum, ileum and colon. At week 26 (5 days after 3rd immunization) lamina propria lymphocytes (LPL) were harvested from the control monkey (group IV) and all vaccinated monkeys (group I-III). SIV-Gag-specific CD8 T cells were detected by Mamu-A*01/Gag181-189 CM9 (CTPYDINQM; Gag_CM9) and SIV-Tat-specific CD8 T cells by Mamu-A*01/Tat 28-35 SL8 (TTPESANL; Tat SL8) tetramer and CD8β and analyzed by flow cytometry. FIG. 5A shows the frequency of tetramer⁺ cells (gag shown in black and tat shown in white) within CD8 gate. Bars represent mean±SE of the percentage tetramer⁺ cells (n=2). FIG. 5B shows dot plots from representative monkeys (M945 (that has been vaccinated with 5 μg of Gp96-Ig-SIV) and control monkey M964 demonstrating binding of Gag CM9 tetramer and Tat SL8 tetramer on CD8 T cells in lamina propria obtained from rectal mucosa, jejunum, ileum and colon. Numbers represent percent of tetramer⁺ cells within CD8 gate. ND-not determined

FIGS. 6A, 6B show that Gp96-Ig-SIV vaccine induces Gag- and Tat-specific CD8 T cells in the intraepithelial compartment At week 26 (5 days after 3rd immunization) intraepithelial lymphocytes (IEL) were harvested from the control monkey (group IV) and all the vaccinated monkeys (group I-III). FIG. 6A: SIV-Gag-specific CD8 T cells were detected by Mamu-A*01/Gag181-189 CM9 (CTPYDINQM; Gag_CM9) and SIV-Tat-specific CD8 T cells by Mamu-A*01/Tat 28-35 SL8 (TTPESANL; Tat SL8) tetramer and CD8β and analyzed by flow cytometry. Numbers represent percent of tetramer⁺ cells within CD8 gate. The individual data for each monkey has been plotted. FIG. 6B shows dot plots from representative monkeys (M945 and M964) demonstrating binding of SIV-gag tetramer on CD8 T cells from intraepithelial compartment (rectal mucosa). Numbers represent percent of Gag-CM9-tetramer⁺ cells within CD8 gate or percent of CD103⁺ cells within tetramer gate.

FIGS. 7A, 7B show that Gp96-Ig-SIV vaccine induced Gag-CM9-specific CD8 T cells in lamina propria and intraepithelial compartment express granzyme B. Lamina propria lymphocytes (LPL) and intraepithelial lymphocytes (IEL) were harvested from the control monkey (group IV) and all the vaccinated monkeys (group I-III) at week 26. LPL (FIG. 7A) and IEL (FIG. 7B) were labeled with anti-human antibodies: CD8β-PC5, CD103-FITC and tetrameric complex (CM9), fixed/permeablized and stained for granzyme B. Frequency of granzyme B⁺ Gag-CM9-specific CD8 T cells is shown. *p<0.05, **p<0.01, ***p<0.001. Dot plots from representative monkeys M945 and M944 demonstrating expression of granzyme B within gated Gag-CM9 tetramer⁺ CD8 T cells. Numbers represent percent of granzyme B⁺ cells within gag-CM9 tetramer gate.

FIGS. 8A-8B show SIV gag-, tat-, env-specific polyfunctional CD8⁺ and CD4⁺ T-cell responses in lamina propria and intraepithelial compartment of Gp96-Ig-SIV vaccinated macaques. At week 26 (5 days after 3rd immunization) lamina propria lymphocytes (LPL) and intraepithelial lymphocytes (IEL) were harvested from the control monkey (group IV) and vaccinated monkeys (group I-III). SIV-specific CD4⁺ and CD8⁺ T-cell responses were detected using pools of 15-meric peptides overlapping by 11 amino acids covering entire Gag, Env, and Pol proteins of SIV mac239. After 6 h peptide stimulation in presence of monensin, cells were stained for the surface antigens with anti-CD8β and anti-CD4 antibodies, permeablized and stained with anti-human IL-2-FITC and anti-human-IFN-γ-APC monoclonal antibodies. FIG. 8A shows the overall polyfunctionality of SIV-specific CD8⁺ and CD4⁺ T-cell responses in freshly isolated rectal lamina propria and (FIG. 8B) intraepithelial cell samples from Mamu-A*01-positive animal M943 are shown as a pie chart. Each colored piece of a pie chart represents proportions of IFN-γ, IL-2 and IFN-γ⁺IL-2⁺ responses within the total number of responding cells. The number in the center of each pie represents the total percentage of cells responding in any way to gag, tat or env stimulation.

FIGS. 9A-9B show that Gfp-OT-1 cells locate to the mucosa after gp96-Ig immunization. Mice received one million gfp-OT-I IV. After 2 days they received four million EG7-gp96-Ig (left) or EG7 (right panels) IP as vaccine. After 5 days, the frequency of gfp-OT-I was analyzed in Peyer's patch lymphocytes (PPL), intraepithelial lymphocytes (IEL), and lamina propria lymphocytes (LPL). FIG. 9A: Representative dot plots of PPL, IEL, and LPL on day 5 after staining for CD8 and analyzing the lymphocyte gate. Numbers in the quadrants represent percent of CD8⁺ or CD8⁺ gfp⁺ OT-1 cells within the lymphocyte gate. FIG. 9B: OT-I expansion expressed as %±s.e.m. of CD8 cells at various sites. The number of mice per group was n=3-6. * P<0.05, * * P<0.01, * * * P<0.001 (compared with EG7 immunization).

FIGS. 10A, 10B show that NIH-3T3-OVA-gp96-Ig cells administered IP mediate strong systemic and mucosal and systemic OT-I expansion. Mice received one million gfp-OT-I IV. After 2 days they received two million NIH-3T3-OVA-gp96-Ig cells (upper panels) or 2 million NIH-3T3-OVA cells (lower panels) IP as vaccine. After 5 days, the frequency of gfp-OT-I was analyzed within intraepithelial lymphocytes (IEL), Peyer's patch lymphocytes (PPL), lamina propria lymphocytes (LPL), peritoneal cavity (PEC), spleen (SPL), and mesenteric lymph nodes (MLNs). FIG. 10A: Representative dot plots of PEC, SPL, MLN, PPL, IEL, and LPL on day 5 after staining for CD8 and analyzing the lymphocyte gate. Numbers in the quadrants represent percent of positive cells within lymphocyte gate. FIG. 10B: Summary of OT-I expansion in 3-6 experiments expressed as %±s.e.m. of CD8 cells at various sites. * P<0.05, * * P<0.01, * * * P<0.001 (compared with 3T3-OVA immunization).

FIGS. 11A-11C. show the mucosal and systemic phenotype of gp96-ova cross-primed OT-I at mucosal and systemic sites. Mice received one million gfp-OT-I IV. After 2 days they received two million NIH-3T3-OVA-gp96-Ig cells IP. After 5 days, the phenotype of gfp-OT-I was analyzed within Peyer's patch lymphocytes (PPL), intraepithelial lymphocytes (IELs) and lamina propria lymphocytes (LPLs), and compared with naive gfp-OT-I (before transfer). FIG. 11A: Each bar represents the mean±s.e. from 4 to 6 mice and (FIG. 11B) numbers represent the percentage of CCR9+ cells among gfp-OT-I cells before and after transfer. FIG. 11C: The overlay histogram shows granzyme B expression in naive gfp-OT-I (dark gray histogram) and gfp-OT-I that had migrated to different mucosal and systemic compartments: PPL (dashed line), IEL (dotted line), LPL (solid line) 5 days after gp96-Ig vaccination. Isotype control is shown in light gray histogram. Cells from different compartments were stained for surface CD8 and than fixed/permeablized and intracellular staining for granzyme B was performed.

FIG. 12: Antigen-specific memory CD8T cells that migrate to spleen or intestinal mucosa after NIH-3T3-ova-gp96-Ig immunization differ in phenotype and function. Mice received one million gfp-OT-I IV. After 2 days they received two million NIH-3T3-OVA-gp96-Ig IP. After 5 days, the phenotype of gfp-OT-I cells in spleen (solid line) and in IEL (dashed line) was compared. To determine granzyme B expression, 5 days after NIH-3T3-OVA-gp96-Ig IP immunization, SPL, IEL and LPL were stained for surface CD8 and than fixed/permeablized and intracellular staining for granzyme B was performed. Granzyme B expression in OT-I cells in spleen (solid line), IEL (dashed line), and LPL (dotted line) was compared. The overlay histogram shows granzyme B expression in gated gfp-OT-I cells and isotype control (gray filled histogram). Representative histograms of 3-6 experiments performed (3-5 mice per experiment). MFI, mean fluorescence intensity; SPL, spleen; PEC, peritoneal exudate cell; IEL, intraepithelial lymphocytes; LPL, lamina propria lymphocytes.

FIGS. 13A-13D: OT-I cells that migrated to the intestinal mucosa after gp96-Ig vaccination express IFN-γ and IL-2, but not IL-17. To detect intracellular cytokine protein accumulation, 5 days after IP NIH-3T3-OVA-gp96-Ig immunization, SPL, IEL, and LPL were incubated with (FIG. 13A) and (FIG. 13B) 20 n M SIINFEKL peptide and 10 ng ml⁻¹. Brefeldin A for 5 h or with (FIG. 13C) 1 μg ml⁻¹ ionomycin, 50 ng ml-15 phorbol 12-myristate 13-acetate, and 2 m M monensin. Cells were first stained for surface anti-CD8 and then fixed/permeabilized, and intracellular staining for IFN-γ, IL-2, or IL-17 was performed. Representative dot plots show gated CD8 cells expressing (FIG. 13A) IFN-γ, (FIG. 13B) IL-2, (FIG. 13C) IL-17, and (FIGS. 13A-13C) gfp-OT-I. Numbers in the quadrants represent percent positive cells within the CD8 gate. FIG. 13D: Frequency of IFN-γ and IL-2 producing OT-I expressed as %±s.e.m. of total OT-I. The number of mice per group was n=3-6. * P<0.05, * P<0.01 (compared with SPL OT-I). SPL, spleen; IEL, intraepithelial lymphocytes; LPL, lamina propria lymphocytes; IFN-γ, interferon-γ; IL-2, interleukin 2.

FIGS. 14A-14D: Intraperitoneal immunization with secreted gp96-Ig increases frequency of CD103+DCs and efficiently upregulates CCR9 on responding T cells in vitro. Mice received two million 3T3-ova-gp96-Ig, 3T3-OVA, or PBS. Peritoneal cells were harvested on days 0, 1, 2 and 5, and stained for CD11c, MHC class II and CD103. FIG. 14A: Absolute numbers of CD11c^{high+} MHC class II^{high+} cells expressing CD103 within PEC cells. Results are the mean±s.e. from three independent experiments (three mice per experiment). * P<0.05, * * P<0.01, * * * P<0.001 (compared with PBS). FIG. 14B: Phenotypic characteristics of CD11c^{high+} MHCII^{high+} cells from PEC 5 days after PBS or 3T3-OVA-gp96 vaccination. FIG. 14C: Sorted CD103⁺ and CD103⁻ DCs from vaccinated (3T3-OVA-gp96) and non-vaccinated (PBS) mice were pulsed with 2 n M SIINFEKL peptide and incubated with CFSE-labeled OT-I cells at the ratio 1:2. CCR9 on responding OT-I cells was assesses after 5 days by flow cytometry. Results are mean and s.e. from two experiments. * P<0.05, * * P<0.01 (compared with CD103⁻ DCs). FIG. 14D: Plots are representative of two independent experiments. PEC, peritoneal cavity; CFSE, carboxyfluorescein diacetate succinimidyl ester; MHC, major histocompatibility complex; PBS, phosphate-buffered saline; DC, dendritic cell.

FIGS. 15A-15D: Priming for generation of gut-tropic T cells after gp96 vaccination is occurring in the peritoneum. CD45.1⁺ mice received two million 3T3-OVA-gp96-Ig IP 2 days after CFSE-labeled CD45.2⁺ OT-I transfer (one million) IV. Peritoneal and mesenteric lymph node (LN) cells were harvested on days 2 and 4, and stained for CD45.2, CD8, and CCR9. FIG. 15A: Representative dot plot/histograms of CCR9 expression and of CFSE dilution on gated CD45.2⁺ CD8T cells. CFSE profile of OT-I cells in the PEC in the absence of gp96 vaccination shown in gray filled histograms. FIG. 15B: Line graph shows the percentage of transferred CFSE-labeled CD45.2⁺ OT-I cells in MLN and PEC at days 2 and 4 that have undergone 0-8 cell divisions as calculated by FlowJo curve-fitting software. Results are mean and s.e. from two experiments. * P<0.05, * * P<0.01, * * * P<0.001 (compared with MLN). FIG. 15C: Bars represent mean±s.e. of the percentage CCR9+OT-I cells (n=3). * * * P<0.001 (compared with MLN). FIG. 15D is a schematic representation of gp96-Ig-induced activation/proliferation and migration of OT-I cells to the intestinal lamina propria after intraperitoneal immunization. PEC, peritoneal cavity; MLN, mesenteric lymph nodes; DC, dendritic cell; CFSE, carboxyfluorescein diacetate succinimidyl ester.

FIG. 16 shows that intraperitoneal immunization with secreted gp96-Ig stimulates more mucosal and systemic immunity compared with subcutaneous and intradermal stimulation, but similar systemic immunity; vaginal and rectal instillation of cells secreting gp96-Ig mediates only mucosal and systemic immunity. Mice received one million purified gfp-OT-I IV. After 2 days two million 3T3-OVA-gp96-Ig cells were injected by different routes: intraperitoneal, subcutaneous, or intradermal; for rectal and vaginal immunization gp96-Ig-secreting cells were instilled. After 5 days the frequency of gfp-OT-I was analyzed in spleen (SPL), mesenteric lymph nodes (MLNs), within intraepithelial lymphocytes (IEL), Peyer's patch lymphocytes (PPL), and lamina propria lymphocytes (LPL). Results are the mean and s.e. of 2-4 experiments (2-4 mice per group). * P<0.05, * * P<0.01 (compared with intraperitoneal immunization).

### DETAILED DESCRIPTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the molecules disclosed herein are not limited to mice but human molecules are preferred, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “induces or enhances an immune response” is meant causing a statistically measurable induction or increase in an immune response over a control sample to which the peptide, polypeptide or protein has not been administered. Preferably the induction or enhancement of the immune response results in a prophylactic or therapeutic response in a subject. Examples of immune responses are increased production of type I IFN, increased resistance to viral and other types of infection by alternate pathogens. The enhancement of immune responses to tumors (anti-tumor responses), or the development of vaccines to prevent tumors or eliminate existing tumors.

The term “active fragment or variant” is meant a fragment that is at least 380 amino acid residues in length and is 100% identical to a contiguous portion of the peptide, polypeptide or protein, or a variant that is at least 90%, preferably 95% identical to a fragment up to and including the full length peptide, polypeptide or protein. A variant, for example, may include conservative amino acid substitutions, as defined in the art, or nonconservative substitutions, providing that at least e.g. 10%, 25%, 50%, 75% or 90% of the activity of the original peptide, polypeptide or protein is retained. Also included are molecules, fragments or variants having post-translational modifications such as sumoylation, phosphorylation glycosylation, splice variants, and the like.

Unless otherwise indicated, the terms “peptide”, “polypeptide” or “protein” are used interchangeably herein, although typically they refer to peptide sequences of varying sizes.

The term “variant,” when used in the context of a polynucleotide sequence, may encompass a polynucleotide sequence related to a wild type gene. This definition may also include, for example, “allelic,” “splice,” “species,” “natural splice” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) or single base mutations in which the polynucleotide sequence varies by one base. The presence of SNPs may be indicative of; for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, cofactors, inhibitors, magnetic particles, and the like.

A “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

An “expression vector” is any genetic element, e.g., a plasmid, chromosome, virus, behaving either as an autonomous unit of polynucleotide replication within a cell. (i.e., capable of replication under its own control) or being rendered capable of replication by insertion into a host cell chromosome, having attached to it another polynucleotide segment, so as to bring about the replication and/or expression of the attached segment. Suitable vectors include, but are not limited to, plasmids, bacteriophages and cosmids. Vectors may contain polynucleotide sequences which are necessary to effect ligation or insertion of the vector into a desired host cell and to effect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors may be capable of directly expressing nucleic acid sequence products encoded therein without ligation or integration of the vector into host cell DNA sequences.

The terms “transformed” or “transfected” are used interchangeably and refer to the process by which exogenous DNA or RNA is transferred or introduced into an appropriate host cell. Transfected host cells include stably transfected cells wherein the inserted DNA is rendered capable of replication in the host cell. Typically, stable transfection requires that the exogenous DNA be transferred along with a selectable marker nucleic acid sequence, such as for example, a nucleic acid sequence that confers antibiotic resistance, which enables the selection of the stable transfectants. This marker nucleic acid sequence may be ligated to the exogenous DNA or be provided independently by simultaneous cotransfection along with the exogenous DNA. Transfected cells also include transiently expressing cells that are capable of expressing the RNA or DNA for limited periods of time. The host cell maybe a prokaryotic or eukaryotic cell. The transfection procedure depends on the host cell being transfected. It can include packaging the polynucleotide in a virus as well as direct uptake of the polynucleotide. Transformation can result in incorporation of the inserted DNA into the genome of the host cell or the maintenance of the inserted DNA within the host cell in plasmid form. Methods of transformation/transfection are well known in the art and include, but are not limited to, direct injection, such as microinjection, viral infection, particularly replication-deficient adenovirus infection, electroporation, lipofection, calcium phosphate-mediated direct uptake and the like.

The term “host cell” generally refers to recombinant cells, prokaryotic or eukaryotic cells and includes any transformable cell which is capable of expressing a protein and can be, or has been, used as a recipient for expression vectors or other transfer DNA.

The term “recombinant cells” refers to cells that have been modified by the introduction of heterologous DNA or RNA. Examples include, but not limited to immune cells, fibroblasts, stem cells, HEK293 and WAS cells. However, any mammalian cell line could be used.

The term “immunoregulatory” or “modulator” is meant a compound, composition or substance that is immunogenic (i.e. stimulates or increases an immune response) or . . . immunosuppressive (i.e. reduces or suppresses an immune response).

“Treating” or “treatment” of a state, disorder or condition includes: (1) Preventing or delaying the appearance of clinical or sub-clinical symptoms of the state, disorder or condition developing in a mammal that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) Relieving the disease, i.e., causing regression of the state, disorder or condition or at least one of its clinical or sub-clinical symptoms. The benefit to a subject to be treated is either statistically significant or at least perceptible to the patient or to the physician.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “therapeutically effective amount” or a “therapeutic amount” is an amount of a therapeutic composition sufficient to produce a measurable response (e.g., a biologically or clinically relevant response in a subject being treated).

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

The terms “patient”, “subject” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

### Vaccines/Compositions

Recent failure of STEP phase 2b clinical trial of adenovirus-5 (Ad5) vectors emphasized the urgent need for the change of the current CD8 T cell vaccine concept. Embodiments of the invention, using HIV/SIV as a model pathogen and an illustrative example herein, are directed to an HIV vaccine that can induce immune responses capable of containing the virus within the mucosa before the establishment of a systemic infection. Heretofore, the majority of T-cell vaccine candidates have not been designed specifically to induce mucosal and systemic T-cell responses (Koff W. C. et al. Replicating viral vectors as HIV vaccines Summary Report from IAVI Sponsored Satellite Symposium, International AIDS Society Conference, Jul. 22, 2007. Biologicals 36, 277-286) and with very few exceptions vaccines are being administered parentally, with immunogenicity assessments and conclusions being drawn from peripheral blood samples in nearly all vaccine trials to date. The vaccines herein, focus on strategies that specifically induce mucosal and systemic immunity.

In one embodiment, a vaccine comprises a heat shock protein, chaperone gp96, based fusion protein vaccine that generates effective anti-tumor immunity in vivo. The data have also advanced the understanding of the mechanism of action of gp96-Ig including the importance of DC and NK cells for the generation of an effective CD8⁺ CTL cell response. These studies identified gp96-peptide complexes as extremely efficient, femto molar pathway of MHC I mediated antigen cross presentation, in generating mucosal and systemic CD8 CTL responses detectable in the mouse intestinal lamina propria and in intraepithelial compartment. These studies provide the first evidence that cell based gp96-Ig secreting vaccines may serve as a potent modality to induce mucosal and systemic immunity. Without wishing to be bound by theory, it was hypothesize that cell-based gp96-Ig vaccines, by prolonged in vivo secretion of gp96-Ig peptide, imitate viral replication and provide immune stimuli comparable to attenuated viruses. In the examples section which follows, it was shown that gp96-Ig transfected, antigen expressing tumor cells secrete gp96-Ig in vivo and stimulate the innate DC and NK as well as adaptive, cognate cellular CD8 CTL immune response and generate specific CD8 memory independent of CD4 help and in the absence of lymph nodes. Because of these unique gp96 properties the gp96-vaccines were evaluated in nonhuman primate model for their safety and immunogenicity. In contrast to previous HIV/SIV vaccines that induce predominant systemic T-cell response and which not only failed to protect patients from disease, but also had little impact on viral load, the vaccines or compositions herein comprising secreted gp96-Ig-SIV vaccine can predominantly induce SIV-specific mucosal and systemic immunity that substantially contribute to neutralization of pathogens at their portal of entry.

While the invention may be applied to any type of immunogen, immunogens of particular interest are those associated with, derived from, or predicted to be associated with an infectious disease, and, as such, may be a bacterium, virus, protozoan, mycoplasma, fungus, yeast, parasite, or prion. For example, but not by way of limitation, the immunogen may be a human immunodeficiency virus 1 or 2 (see below), a herpes virus such as herpes simplex or herpes zoster, other retroviruses, such Human T-cell Lymphotropic Virus, a hepatitis virus, an influenza virus, a rhinovirus, respiratory syncytial virus, cytomegalovirus, adenovirus, Mycoplasma pneumoniae, a bacterium of the genus Salmonella, Staphylococcus, Streptococcus, Enterococcus, Clostridium, Escherichia, Klebsiella, Vibrio, Mycobacterium, amoeba, a malarial parasite, Trypanosoma cruzi, etc. In further embodiments, the immunogen may be associated with neoplastic diseases, including but not limited to a sarcoma, a lymphoma, a leukemia, or a carcinoma, and in particular, with melanoma, carcinoma of the breast, carcinoma of the prostate, ovarian carcinoma, carcinoma of the cervix, colon carcinoma, carcinoma of the lung, glioblastoma, astrocytoma, etc. Any one or more antigens from the pathogen can be used as part of the vaccine. In addition, some of the antigens can be derived from different pathogens, or different diseases such as, colon cancer.

Immunogens may be obtained by isolation directly from a neoplasm, an infected cell, a specimen from an infected subject, a cell culture, or an organism culture, or may be synthesized by chemical or recombinant techniques. Suitable antigenic peptides, particularly for use in a hybrid antigen, for use against viruses, bacteria and the like can be designed by searching through their sequences for MHC class I restricted peptide epitopes containing HLA binding sequences.

In a preferred embodiment, a method of inducing both mucosal and systemic and systemic immunity comprising administering to a patient in need thereof; a therapeutically effective amount of a vaccine having at least one heat shock protein, at least one immunogen from one or more pathogens or diseases, fragments variants, derivatives, mutants, or combinations thereof.

In preferred embodiments, a method of inducing HIV/SIV antigen specific mucosal and systemic immunity and systemic immunity in vivo, comprises administering to a patient in need thereof, a therapeutically effective amount of antigen comprising a heat shock protein, or fragments thereof, such as for example, gp96. Preferably the gp96 is secreted (gp96-Ig). The heat shock protein is not just limited to gp96 but extends to all other heat shock proteins. Heat shock proteins are among the most highly conserved proteins in existence. For example, DnaK, the hsp70 from E. coli has about 50% amino acid sequence identity with hsp70 proteins from excoriates (Bardwell, et al., 1984, Proc. Natl. Acad. Sci. 81:848-852). The hsp60 and hsp90 families also show similarly high levels of intrafamilies conservation (Hickey, et al., 1989, Mol. Cell. Biol. 9:2615-2626; Jindal, 1989, Mol. Cell. Biol. 9:2279-2283). In addition, the hsp60, hsp70 and hsp90 families are composed of proteins that are related to the stress proteins in sequence, for example, having greater than 35% amino acid identity, but whose expression levels are not altered by stress. Therefore it is contemplated that the definition of heat shock protein or stress protein, as used herein, embraces other proteins, muteins, analogs, and variants thereof having at least 35% to 55%, preferably 55% to 75%, and most preferably 75% to 85% amino acid identity with members of the three families whose expression levels in a cell are enhanced in response to a stressful stimulus.

In another preferred embodiment, administration of the gp96-Ig composition to a patient in need thereof induces an HIV/SIV antigen specific mucosal and systemic immune response comprising induction of an antigen specific T cell immune response. Preferably, the antigen specific T cell response is polyspecific comprising CD8, CD4 T cells, innate dendritic cell, natural killer cells (NK), and memory CD8⁺ T cells.

In another preferred embodiment, the HIV/SIV antigen comprises: an isolated cell having a plasmid encoding gp96-Ig, HIV/SIV antigens retanef (Rev-Tat-Nef), gag, gp160, fragments, variants, mutants, derivatives or combinations thereof. The retanef preferably comprises at least one of Rev, Tat, Nef, fragments, variants, mutants, derivatives or combinations thereof.

In another preferred embodiment, the isolated cell expresses endogenous, membrane bound, secreted or combinations thereof of at least one of the molecules comprising: gp96, retanef, gag, gp160, fragments, variants, mutants, derivatives or combinations thereof.

In another preferred embodiment, the cell comprises autologous, syngeneic, heterologous, xenogeneic cells, cell lines, or combinations thereof.

In another preferred embodiment, a method of preventing HIV in a patient at risk of being infected with HIV, or treating a patient, infected with HIV, comprises administering to the patient in need thereof, a therapeutically effective amount of antigen comprising gp96, wherein the antigen induces an HIV/SIV antigen specific mucosal and systemic immunity comprising an antigen specific T cell immune response.

In another preferred embodiment, the HIV/SIV antigen induces immune cells comprising central memory T cells (T_{CM}; CD95⁺ CD28⁺), effector memory T cells (T_{EM}; CD95⁺ CD28⁻) or naive T cells (CD95^{low} CD28^{int}).

In another preferred embodiment, an isolated nucleic acid encoding at least one molecule comprising: gp96-Ig, HIV/SIV antigens retanef (Rev-Tat-Net), gag, gp160, fragments, variants, mutants, derivatives or combinations thereof.

In another preferred embodiment, an isolated nucleic acid encoding at least one molecule comprising: gp96-Ig, an immunogen (e.g. tumor antigen, antigens associated with infectious organisms, etc.), fragments, variants, mutants, derivatives or combinations thereof.

In another preferred embodiment, the expression vector is a bicistronic vector. In one aspect, the vector comprises an SV40 promoter, however, any type of promoter that is functional in different cell types can be used, including tissue specific promoters. Examples of promoters useful to practice the present invention, include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metallothionein.

In one preferred embodiment, the encoded molecules are endogenous, membrane bound, secreted or combinations thereof. Preferably, the molecules are secreted.

In another preferred embodiment, a fusion protein comprising at least one of: gp96-Ig, HIV/SIV antigens retanef (Rev-Tat-Nef), gag, gp160, fragments, variants, mutants, derivatives or combinations thereof.

In another preferred embodiment, a fusion protein comprising at least one of: gp96-Ig, immunogen, fragments, variants, mutants, derivatives or combinations thereof. Preferably the gp96 is fused or linked to the immunogen and the molecule is secreted. The molecule can be encoded by an expression vector in a cell, preferably a mammalian cell. The cell can be obtained from a patient, which is cultured ex-vivo; the cell is contacted with the expression vector; cells producing the molecule are then re-infused into the patient, via any mode, such as i.v. i.p. etc.

In another preferred embodiment, an isolated cell comprising a nucleic acid molecule encoding at least one or more of gp96-Ig, HIV/SIV antigens retanef (Rev-Tat-Nef), gag, gp160, fragments, variants, mutants, derivatives or combinations thereof.

In another preferred embodiment, a method of inducing Human Immunodeficiency Virus (HIV) specific immune response in vivo or in vitro, comprising: administering to the patient in need thereof, a therapeutically effective amount of an HIV/SIV specific molecule or immunogen comprising gp96.

Preferably, the immunogen comprising gp96 induces an HIV/SIV antigen specific mucosal and systemic immunity comprising an antigen specific T cell immune response. The antigen specific T cell response is preferably, polyspecific comprising CD8⁺ and CD4⁺ T cells, wherein the T cells co-express and produce IFNγ and IL-2. In another embodiment, the HIV/SIV antigen specific mucosal and systemic immunity further comprises innate dendritic cell, natural killer cells (NK), CD103⁺ cells, CD8⁺ CD103⁺ T cells, and/or memory CD8+ T cells. Preferably, the memory cells comprise central memory T cells (T_{CM}; CD95⁺ CD28⁺), effector memory T cells (T_{EM}; CD95⁺ CD28⁻) or naive T cells (CD95^{low} CD28^{int}).

As discussed above, the heat shock protein can be from any family of hsp and the immunogen is selected from the disease of interest. The illustrative examples herein described the retanef, gag for HIV. The compositions comprising hsp can be fused, linked, covalently or noncovalently bound to the antigenic molecules or immunogens and are administered to elicit an effective specific immune response to the molecules. In accordance with the methods described herein, the hsp-antigenic molecule complexes are preferably purified in the range of 60 to 100 percent of the total mg protein, or at least 70%, 80% or 90% of the total mg protein. In another embodiment, the hsp-antigenic molecule complexes are purified to apparent homogeneity, as assayed by sodium dodecyl sulfate-polyacrylamide gel electrophoresis.

In a preferred embodiment, the complexes of hsp70, hsp90 and gp96 with peptides are prepared and purified postoperatively from, for example, tumor cells obtained from the cancer patient or cells from an infected patient, such as for example, an HIV infection.

In accordance with the methods described herein, immunogenic or antigenic peptides that are endogenously complexed to hsps or MHC antigens can be used as antigenic molecules. For example, such peptides may be prepared that stimulate cytotoxic T cell responses against different tumor antigens (e.g., tyrosinase, gp100, melan-A, gp75, mucins, etc.) and viral proteins including, but not limited to, proteins of immunodeficiency virus type I (HIV-I), human immunodeficiency virus type II (HIV-II), hepatitis type A, hepatitis type B, hepatitis type C, influenza, Varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus and polio virus. In the embodiment wherein the antigenic molecules are peptides linked to hsps in vive, the complexes can be isolated from cells, or alternatively, produced in vitro from purified preparations each of hsps and antigenic molecules.

In another specific embodiment, antigens of cancers (e.g., tumors) or infectious agents (e.g., viral antigen, bacterial antigens, etc.) can be obtained by purification from natural sources, by chemical synthesis, or recombinantly, and, through in vitro procedures such as those described in detail in the examples which follow.

In an embodiment wherein it is desired to treat a patient having an infectious disease, the methods in the examples section describe the hsp-peptide complexes from cells infected with an infectious organism, e.g., of a cell line or from a patient. Such infectious organisms include but are not limited to, viruses, bacteria, protozoa, fungi, and parasites.

In alternative embodiments, the antigenic peptides and/or components can be eluted from hsp-complexes either in the presence of ATP or low pH. These experimental conditions may be used to isolate peptides and/or antigenic components from cells which may contain potentially useful antigenic determinants. Once isolated, the amino acid sequence of each antigenic peptide may be determined using conventional amino acid sequencing methodologies. Such antigenic molecules can then be produced by chemical synthesis or recombinant methods, purified, and complexed to hsps in vitro.

Similarly, it has been found that potentially immunogenic peptides may be eluted from MHC-peptide complexes using techniques well known in the art (Falk, K. et al., 1990 Nature 348:248-251; Elliott, T., et al., 1990, Nature 348:195-197; Falk, K., et al., 1991, Nature 351:290-296).

Antigens or antigenic portions thereof can be selected for use as antigenic molecules, for complexing to hsps, from among those known in the art or determined by immunoassay to be able to bind to antibody or MHC molecules (antigenicity) or generate immune response (immunogenicity). To determine immunogenicity or antigenicity by detecting binding to antibody, various immunoassays known in the art can be used, including but not limited to competitive and non-competitive assay systems using techniques such as radioimmunoassays, ELISA (enzyme linked immunosorbent assay), “sandwich” immunoassays, immunoradiometric assays, gel diffusion precipitin reactions, immunodiffusion assays, in vivo immunoassays (using colloidal gold, enzyme or radioisotope labels, for example), Western blots, immunoprecipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are envisioned for use. In one embodiment for detecting immunogenicity, T cell-mediated responses can be assayed by standard methods, e.g., in vitro cytoxicity assays or in vivo delayed-type hypersensitivity assays.

Potentially useful antigens or derivatives thereof for use as antigenic molecules can also be identified by various criteria, such as the antigen's involvement in neutralization of a pathogen's infectivity (wherein it is desired to treat or prevent infection by such a pathogen) (Norrby, 1985, Summary, in Vaccines 85, Lerner, et al. (eds.), Cold Spring Harbor Laboratory, Cold Spring Harbor, N.Y., pp. 388-389), type or group specificity, recognition by patients' antisera or immune cells, and/or the demonstration of protective effects of antisera or immune cells specific for the antigen. In addition, where it is desired to treat or prevent a disease caused by pathogen, the antigen's encoded epitope should preferably display a small or no degree of antigenic variation in time or amongst different isolates of the same pathogen.

Preferably, where it is desired to treat or prevent cancer, known tumor-specific antigens or fragments or derivatives thereof are used. For example, such tumor specific or tumor-associated antigens include but are not limited to KS 1/4 pan-carcinoma antigen (Perez and Walker, 1990, J. Immunol. 142:3662-3667; Bumal, 1988, Hybridoma 7(4):407-415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63) and prostate specific membrane antigen.

In a specific embodiment, an antigen or fragment or derivative thereof specific to a certain tumor is selected for complexing to hsp and subsequent administration to a patient having that tumor.

Preferably, where it is desired to treat or prevent viral diseases, molecules comprising epitopes of known viruses are used. For example, such antigenic epitopes may be prepared from viruses including, but not limited to, hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, human immunodeficiency virus type I (HIV-I), and human immunodeficiency virus type II (HIV-II).

Preferably, where it is desired to treat or prevent bacterial infections, molecules comprising epitopes of known bacteria are used. For example, such antigenic epitopes may be prepared from bacteria including, but not limited to, mycobacteria rickettsia, mycoplasma, neisseria and legionella.

Preferably, where it is desired to treat or prevent protozoal infections, molecules comprising epitopes of known protozoa are used. For example, such antigenic epitopes may be prepared from protozoa including, but not limited to, leishmania, kokzidioa, and trypanosoma.

Preferably, where it is desired to treat or prevent parasitic infections, molecules comprising epitopes of known parasites are used. For example, such antigenic epitopes may be from parasites including, but not limited to, chlamydia and rickettsia.

Immune System:

Immune systems are classified into two general systems, the “innate” or “primary” immune system and the “acquired/adaptive” or “secondary” immune system. It is thought that the innate immune system initially keeps the infection under control, allowing time for the adaptive immune system to develop an appropriate response. Studies have suggested that the various components of the innate immune system trigger and augment the components of the adaptive immune system, including antigen-specific B and T lymphocytes (Kos, Immunol. Res. 1998, 17:303; Romagnani, Immunol. Today. 1992, 13: 379; Banchereau and Steinman, Nature. 1988, 392:245).

A primary immune response refers to an innate immune response that is not affected by prior contact with the antigen. The main protective mechanisms of primary immunity are the skin (protects against attachment of potential environmental invaders), mucous (traps bacteria and other foreign material), gastric acid (destroys swallowed invaders), antimicrobial substances such as interferon (IFN) (inhibits viral replication) and complement proteins (promotes bacterial destruction), fever (intensifies action of interferons, inhibits microbial growth, and enhances tissue repair), natural killer (NK) cells (destroy microbes and certain tumor cells, and attack certain virus infected cells), and the inflammatory response (mobilizes leukocytes such as macrophages and dendritic cells to phagocytose invaders).

Some cells of the innate immune system, including macrophages and dendritic cells (DC), function as part of the adaptive immune system as well by taking up foreign antigens through pattern recognition receptors, combining peptide fragments of these antigens with major histocompatibility complex (MHC) class I and class II molecules, and stimulating naive CD8⁺ and CD4⁺ T cells respectively (Banchereau and Steinman, supra; Holmskov et al., Immunol. Today. 1994, 15:67; Ulevitch and Tobias Annu. Rev. Immunol. 1995, 13:437). Professional antigen-presenting cells (APCs) communicate with these T cells, leading to the differentiation of naive CD4⁺ T cells into T-helper 1 (Th1) or T-helper 2 (Th2) lymphocytes that mediate cellular and humoral immunity, respectively (Trinchieri Annu. Rev. Immunol. 1995, 13:251; Howard and O'Garra, Immunol. Today. 1992, 13:198; Abbas et al., Nature. 1996, 383:787; Okamura et al., Adv. Immunol. 1998, 70:281; Mosmann and Sad, Immunol. Today. 1996, 17:138; O'Garra Immunity. 1998, 8:275).

A secondary immune response or adaptive immune response may be active or passive, and may be humoral (antibody based) or cellular that is established during the life of an animal, is specific for an inducing antigen, and is marked by an enhanced immune response on repeated encounters with said antigen. A key feature of the T lymphocytes of the adaptive immune system is their ability to detect minute concentrations of pathogen-derived peptides presented by MHC molecules on the cell surface. Upon activation, naïve CD4 T cells differentiate into one of at least two cell types, Th1 cells and Th2 cells, each type being characterized by the cytokines it produces. “Th1 cells” are primarily involved in activating macrophages with respect to cellular immunity and the inflammatory response, whereas “Th2 cells” or “helper T cells” are primarily involved in stimulating B cells to produce antibodies (humoral immunity). CD4 is the receptor for the human immunodeficiency virus (HIV). Effector molecules for Th1 cells include, but are not limited to, IFN-γ, GM-CSF, TNF-α, CD40 ligand, Fas ligand, IL-3, TNF-β, and IL-2. Effector molecules for Th2 cells include, but are not limited to, IL-4, IL-5, CD40 ligand, IL-3, GS-CSF, IL-10, TGF-β, and eotaxin. Activation of the Th1 type cytokine response can suppress the Th2 type cytokine response, and reciprocally, activation of the Th2 type cytokine response can suppress the Th1 type response. Thus, the immune response is “polarized” toward a Th1 or Th2 response.

In adaptive immunity, adaptive T and B cell immune responses work together with innate immune responses. The basis of the adaptive immune response is that of clonal recognition and response. An antigen selects the clones of cell which recognize it, and the first element of a specific immune response must be rapid proliferation of the specific lymphocytes. This is followed by further differentiation of the responding cells as the effector phase of the immune response develops. In T-cell mediated non-infective inflammatory diseases and conditions, immunosuppressive drugs inhibit T-cell proliferation and block their differentiation and effector functions.

The phrase “T cell response” means an immunological response involving T cells. The T cells that are “activated” divide to produce memory T cells or cytotoxic T cells. The cytotoxic T cells bind to and destroy cells recognized as containing the antigen. The memory T cells are activated by the antigen and thus provide a response to an antigen already encountered. This overall response to the antigen is the T cell response.

“Cells of the immune system” or “immune cells”, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhan's cells, stem cells, dendritic cells, peripheral blood mononuclear cells, tumor-infiltrating (TIL) cells, gene modified immune cells including hybridomas, drug modified immune cells, antigen presenting cells and derivatives, precursors or progenitors of the above cell types.

“Immune effector cells” refers to cells, and subsets thereof; e.g. Treg, Th1, Th2, capable of binding an antigen and which mediate an immune response selective for the antigen. These cells include, but are not limited to, T cells (T lymphocytes), B cells (B lymphocytes), antigen presenting cells, such as for example dendritic cells, monocytes, macrophages; myeloid suppressor cells, natural killer (NK) cells and cytotoxic T lymphocytes (CTLs), for example CTL lines, CTL clones, and CTLs from tumor, inflammatory, or other infiltrates.

In a preferred embodiment, a method of inducing an immune response to a vaccine comprises administering to a patient in need thereof, a therapeutically effective amount of an hsp-immunogen vaccine to induce an antigen specific immune response. Preferably, the molecule is a secreted molecule.

In a preferred embodiment, the antigen specific immune response to the vaccine is regulated, preferably up-regulated. The enhancement of the immune response to a vaccine or other antigenic stimulant can be measured by any conventional method, such as for example proliferation assays, cytokine secretion, types of cytokines secreted, cytotoxic T lymphocyte assays, ELISAS, RIA and the like. The enhanced immune response can also be detected by monitoring the treatment. In the cases of viral infection, plaque assays, viral titers etc., can be used to monitor the clearance of the virus.

In another preferred embodiment, mucosal and systemic immune responses are modulated by administration of a composition comprising an hsp linked to one or more immunogens. The molecule is preferably a secreted molecule and can be administered either alone as an expression vector or in the context of a cell comprising the vector which encodes the desired molecule. For example, the immunogen comprises one or more antigens derived from immunogenic or antigenic peptides. For example, such peptides may be prepared that stimulate cytotoxic T cell responses against different tumor antigens (e.g., tyrosinase, gp100, melan-A, gp75, mucins, etc.) and viral proteins and/or other pathogens including, but not limited to, antigens of human immunodeficiency viruses, such as HIV-1 and HIV-2, polio viruses, hepatitis A virus, human coxsackie viruses, rhinoviruses, echoviruses, equine encephalitis viruses, rubella viruses, dengue viruses, encephalitis viruses, yellow fever viruses, coronaviruses, vesicular stomatitis viruses, rabies viruses, Ebola viruses, parainfluenza viruses, mumps virus, measles virus, respiratory syncytial virus, influenza viruses, Hantaan viruses, bunga viruses, hemorrhagic fever viruses, reoviruses, orbiviruses, rotaviruses, Hepatitis B virus, parvoviruses, papilloma viruses, polyoma viruses, adenoviruses), herpes simplex virus (HSV) 1 and 2, varicella zoster virus, cytomegalovirus (CMV), variola viruses, vaccinia viruses, pox viruses, African swine fever virus, the unclassified agent of delta hepatitis, the agents of non-A, non-B hepatitis; infectious bacteria like: Helicobacter pylori, Borrelia burgdorferi, Legionella pneumophila, Mycobacterium tuberculosis, Mycobacterium bovis (BCG), Mycobacterium avium, Mycobacterium intracellulare, Staphylococcus aureus, Neisseria gonorrhoeae, Neisseria meningitidis, Listeria monocytogenes, Streptococcus pyogenes, Streptococcus pneumoniae, Haemophilus influenzae, Moraxella catharralis, Klebsiella pneumoniae, Bacillus anthracis, Corynebacterium diphtheriae, Clostridium perfringens, Clostridium tetani, Enterobacter aerogenes, Klebsiella pneumoniae, Pasturella multocida, and Treponema pallidum; infectious fungi like: Cryptococcus neoformans, Histoplasma capsulatum, Coccidioides immitis, Blastomyces dermatitidis, Candida albicans; and infectious protists like, for example: Plasmodium falciparum, Trypanosoma cruzi, Leishmania donovani and Toxoplasma gondii; as well as infectious fungi such as those causing e.g., histoplasmosis, candidiasis, cryptococcosis, blastomycosis and cocidiodomycosis; as well as Candida spp. (i.e., C. albicans, C parapsilosis, C. krusei, C. glabrata, C. tropicalis, or C. lusitaniaw); Torulopus spp. (i.e., T. glabrata); Aspergillus spp. (i.e., A. fumigalus), Histoplasma spp. (i.e., H. capsulatum); Cryptococcus spp. (i.e., C. neoformans); Blastomyces spp. (i.e., B. dermatilidis); Fusarium spp.; Trichophyton spp., Pseudallescheria boydii, Coccidioides immits, and Sporothrix schenckii, and; as well as human tumoral cells. In the embodiment wherein the antigenic molecules are peptides noncovalently complexed to hsps in vivo, the complexes can be isolated from cells, or alternatively, produced in vitro from purified preparations each of hsps and antigenic molecules.

Antigens or antigenic portions thereof can be selected for use as antigenic molecules, for association with hsps, from among those known in the art or determined by immunoassay to be able to bind to antibody or MHC molecules (antigenicity) or generate immune response (immunogenicity) as described above. In preferred embodiments, the vaccine stimulates the mucosal and systemic immune response and systemic immune response. This is especially important in cases such as for example, HIV whereby the point of entry is usually via mucosa. The regulation of the mucosal and systemic immune response is also important in those cases where the immune system is associated with the disease. Examples include, colitis, Crohn's disease, inflammatory bowel diseases, arthritis, autoimmune diseases or disorders, allergies, allergic reactions, asthma, lung inflammation and the like.

The mucosal and systemic immune system, consisting of lymphoid tissues associated with the lacrimal, salivary, gastrointestinal, respiratory and urogenital tracts and lactating breasts, quantitatively contains the majority of the lymphoid tissue of the body. There are a number of important features of the gastrointestinal mucosal and systemic immune system: the mucosal and systemic immune system contains specialized structures, such as the Peyer's patches, where immune responses are likely to be initiated; there is a pattern of relatively specific recirculation of lymphoid cells to the mucosa, known as mucosal and systemic homing; subsets of lymphoid cells, particularly IgA B cells and memory T cells, predominate at mucosal and systemic surfaces; and the predominant mucosal and systemic immunoglobulin, secretory IgA, is particularly well adapted to host defense at mucosal and systemic surfaces. These elements of the gastrointestinal mucosal and systemic immune system function together to generate an immune response which on the one hand protects the host from harmful pathogens, but on the other hand is tolerant of the ubiquitous dietary antigens and normal microbial flora.

### Formulations

The invention contemplates delivery of the gp96-Ig molecules comprising; nucleic acids, polypeptides, peptides, vectors, cells comprising gp96-Ig nucleic acids or polypeptides, splice variants and the like. Delivery of polypeptides and peptides can be accomplished according to standard vaccination protocols which are well known in the art.

In another preferred embodiment, a vector comprises an hsp-immunogen such as for example, gp96-Ig polynucleotide, natural splice variants, deletions, variants, mutants or active fragments thereof.

A number of vectors are known to be capable of mediating transfer of gene products to mammalian cells, as is known in the art and described herein. A “vector” (sometimes referred to as gene delivery or gene transfer “vehicle”) refers to a macromolecule or complex of molecules comprising a polynucleotide to be delivered to a host cell, either in vitro or in vivo. The polynucleotide to be delivered may comprise a coding sequence of interest in gene therapy. Vectors include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell. Vectors can also comprise other components or functionalities that further modulate gene delivery and/or gone expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector comprising one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation). Modified viral vectors in which a polynucleotide to be delivered is carried on the outside of the viral particle have also been described (see, e.g., Curiel, D T, et al. PNAS 88: 8850-8854, 1991).

Suitable nucleic acid delivery systems include viral vector, typically sequence from at least one of an adenovirus, adenovirus-associated virus (AAV), helper-dependent adenovirus, retrovirus, or hemagglutinating virus of Japan-liposome (HVJ) complex. Preferably, the viral vector comprises a strong eukaryotic promoter operably linked to the polynucleotide e.g., a cytomegalovirus (CMV) promoter.

Additionally preferred vectors include viral vectors, fusion proteins and chemical conjugates. Retroviral vectors include Moloney murine leukemia viruses and HIV-based viruses. One preferred HIV-based viral vector comprises at least two vectors wherein the gag and pol genes are from an HIV genome and the env gene is from another virus. DNA viral vectors are preferred. These vectors include pox vectors such as orthopox or avipox vectors, herpesvirus vectors such as a herpes simplex I virus (HSV) vector [Geller, A. I. et al., J. Neurochem, 64: 487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A.: 90 7603 (1993); Geller, A. I., et al., Proc Natl. Acad. Sci USA: 87:1149 (1990)], Adenovirus Vectors [LeGal LaSalle et al., Science, 259:988 (1993); Davidson, et al., Nat. Genet. 3: 219 (1993); Yang, et al., J. Virol. 69: 2004 (1995)] and Adeno-associated Virus Vectors [Kaplitt, M. G., et al., Nat. Genet. 8:148 (1994)].

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors may be an indication for some invention embodiments. The adenovirus vector results in a shorter term expression (e.g., less than about a month) than adeno-associated virus, in some embodiments, may exhibit much longer expression. The particular vector chosen will depend upon the target cell and the condition being treated. The selection of appropriate promoters can readily be accomplished. Preferably, one would use a high expression promoter. An example of a suitable promoter is the 763-base-pair cytomegalovirus (CMV) promoter. The Rous sarcoma virus (RSV) (Davis, et al., Hum Gene Ther 4:151 (1993)) and MMT promoters may also be used. Certain proteins can expressed using their native promoter. Other elements that can enhance expression can also be included such as an enhancer or a system that results in high levels of expression such as a tat gene and tar element. This cassette can then be inserted into a vector, e.g., a plasmid vector such as, pUC19, pUC118, pBR322, or other known plasmid vectors, that includes, for example, an E. coli origin of replication. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely effect the metabolism of the organism being treated. The cassette can also be bound to a nucleic acid binding moiety in a synthetic delivery system, such as the system disclosed in WO 95/22618.

If desired, the polynucleotides of the invention may also be used with a microdelivery vehicle such as cationic liposomes and adenoviral vectors. For a review of the procedures for liposome preparation, targeting and delivery of contents, see Mannino and Gould-Fogerite, BioTechniques, 6:682 (1988). See also, Feigner and Holm, Bethesda Res. Lab. Focus, 11(2):21 (1989) and Maurer, R. A., Bethesda Res. Lab. Focus, 11(2):25 (1989).

Replication-defective recombinant adenoviral vectors, can be produced in accordance with known techniques. See, Quantin, et al., Proc. Natl. Acad. Sci. USA, 89:2581-2584 (1992); Stratford-Perricadet, et al., J. Clin. Invest., 90:626-630 (1992); and Rosenfeld, et al., Cell, 68:143-155 (1992).

Another delivery method is to use single stranded DNA producing vectors which can produce the gp96-Ig intracellularly. See for example, Chen et al, BioTechniques, 34: 167-171 (2003), which is incorporated herein, by reference, in its entirety.

Expression of the hsp-immunogen molecules may be controlled by any promoter/enhancer element known in the art, but these regulatory elements must be functional in the host selected for expression. Promoters which may be used to control gp96-Ig gene expression include, but are not limited to, cytomegalovirus (CMV) promoter (U.S. Pat. Nos. 5,385,839 and 5,168,062), the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al., Nature 296:39-42, 1982); prokaryotic expression vectors such as the β-lactamase promoter (Villa-Kamaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., Cell 38:639-646, 1984; Ornitz et al., Cold Spring Harbor Symp. Quant. Biol. 50:399-409, 1986; MacDonald, Hepatology 7:425-515, 1987); insulin gene control region which is active in pancreatic beta cells (Hanahan, Nature 315:115-122, 1985), immunoglobulin gene control region which is active in lymphoid cells (Grosschedl et al., Cell 38:647-658, 1984; Adames et al., Nature 318:533-538, 1985; Alexander et al., Mol. Cell. Biol. 7:1436-1444, 1987), mouse mammary tumor virus control region which is active in testicular, breast, lymphoid and mast cells (Leder et al., Cell 45:485-495, 1986), albumin gene control region which is active in liver (Pinkert et al., Genes and Devel. 1:268-276, 1987), alpha-fetoprotein gene control region which is active in liver (Krumlauf et al., Mol. Cell. Biol. 5:1639-1648, 1985; Hammer et al., Science 235:53-58, 1987), alpha 1-antitrypsin gene control region which is active in the liver (Kelsey et al., Genes and Devel. 1: 161-171, 1987), beta-globin gene control region which is active in myeloid cells (Mogram et al., Nature 315:338-340, 1985; Kollias et al., Cell 46:89-94, 1986), myelin basic protein gene control region which is active in oligodendrocyte cells in the brain (Readhead et al., Cell 48:703-712, 1987), myosin light chain-2 gene control region which is active in skeletal muscle (Sani, Nature 314:283-286, 1985), and gonadotropic releasing hormone gene control region which is active in the hypothalamus (Mason et al., Science 234:1372-1378, 1986).

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Yeast expression systems can also be used according to the invention to express TNFR25. For example, the non-fusion pYES2 vector (XbaI, SphI, Shol, Notd, GstXI, EcoRI, BstXI, BamH1, SacI, KpnI, and HindIII cloning sites; Invitrogen) or the fusion pYESHisA, B, C (XbaI, SphI, Shol, NotI, BstXI, EcoRI, BamH1, SacI, KpnI, and HindIII cloning sites, N-terminal peptide purified with ProBond resin and cleaved with enterokinase; Invitrogen), to mention just two, can be employed according to the invention. A yeast two-hybrid expression system can be prepared in accordance with the invention.

One preferred delivery system is a recombinant viral vector that incorporates one or more of the polynucleotides therein, preferably about one polynucleotide. Preferably, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

In a preferred embodiment, a composition of the invention is administered to a patient via immunization routes. For example, intra-venously, intra-muscularly, intra-peritoneally, and the like. Preferably, the immunization induces a mucosal and systemic immune response, systemic immune response.

In the case of polynucleotides or oligonucleotides, the delivery of the nucleic acid, e.g. encoding gp96-Ig, can be accomplished by ex vivo methods, i.e. by removing a cell from a subject, genetically engineering the cell to include the nucleic acid, and reintroducing the engineered cell into the subject. One example of such a procedure is outlined in U.S. Pat. No. 5,399,346. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

In another preferred embodiment, an isolated cell expresses hsp-immunogens, for example, gp96-Ig molecules. The cell can be autologous, syngeneic, xenogeneic et, stem cell, immune cell, mucosal and systemic cell and the like.

In another embodiment, the vaccines can be administered to autologous cells, allow the cells to expand and then re-infuse the cells into the patient.

The compositions can be administered in a pharmaceutical composition, as a polynucleotide in a vector, liposomes, nucleic acids peptides and the like.

In another preferred embodiment, the compositions can be administered with one or more or additional pharmacologically active agents. As used herein, the term “pharmacologically active agent” refers to any agent, such as a drug, capable of having a physiologic effect (e.g., a therapeutic or prophylactic effect) on prokaryotic or eukaryotic cells, in vivo or in vitro, including, but without limitation, chemotherapeutics, anti-virals, toxins, radiotherapeutics, radiosensitizing agents, gene therapy vectors, antisense nucleic acid constructs or small interfering RNA, imaging agents, diagnostic agents, agents known to interact with an intracellular protein, polypeptides, and polynucleotides.

The additional pharmacologically active agent can be selected from a variety of known classes of drugs, including, for example, analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

The additional pharmacologically active agent need not be a therapeutic agent. For example, the agent may be cytotoxic to the local cells to which it is delivered but have an overall beneficial effect on the subject. Further, the agent may be a diagnostic agent with no direct therapeutic activity per se, such as a contrast agent for bioimaging.

In another preferred embodiment, a gp96-Ig polynucleotide or peptide are labeled with a detectable marker, such as for example, fluorescent markers (e.g. GFP, RFP etc) or radiolabels.

“Detectable moiety” or a “label” refers to a composition detectable by spectroscopic, photochemical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

### Administration of Compositions

The compositions of the present invention may be administered in conjunction with one or more additional active ingredients, pharmaceutical compositions, or other vaccines. The therapeutic agents of the present invention may be administered to an animal, preferably a mammal, most preferably a human.

The pharmaceutical formulations and vaccines may be for administration by oral (solid or liquid), parenteral (intramuscular, intraperitoneal, intravenous (IV) or subcutaneous injection), transdermnal (either passively or using ionophoresis or electroporation), transmucosal and systemic (nasal, vaginal, rectal, or sublingual), or inhalation routes of administration, or using bioerodible inserts and can be formulated in dosage forms appropriate for each route of administration.

The agents may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

In some embodiments, the compositions or vaccines are administered by pulmonary delivery. The composition or vaccine is delivered to the lungs of a mammal while inhaling and traverses across the lung epithelial lining to the blood stream [see, e.g., Adjei, at al. Pharmaceutical Research 1990; 7:565 569; Adjei, et al. Int. J. Pharmaceutics 1990; 63:135 144 (leuprolide acetate); Braquet, et al. J. Cardiovascular Pharmacology 1989; 13(sup5):143 146 (endothelin-1); Hubbard, et al. (1989) Annals of Internal Medicine, Vol. III, pp. 206 212 (α1 antitrypsin); Smith, et al. J. Clin. Invest. 1989; 84:1145-1146 (α 1-proteinase); Oswein, et al. “Aerosolization of Proteins”, 1990; Proceedings of Symposium on Respiratory Drug Delivery II Keystone, Colorado (recombinant human growth hormone); Debs, et al. J. Immunol. 1988; 140:3482 3488 (interferon γ and tumor necrosis factor α); and U.S. Pat. No. 5,284,656 to Platz, et al. (granulocyte colony stimulating factor). A method and composition for pulmonary delivery of drugs for systemic effect is described in U.S. Pat. No. 5,451,569 to Wong, et al. See also U.S. Pat. No. 6,651,655 to Licalsi et al.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). All such devices require the use of formulations suitable for the dispensing of the therapeutic agent. Typically, each formulation is specific to the type of device employed and may involve the use of an appropriate propellant material, in addition to the usual diluents, adjuvants, surfactants and/or carriers useful in therapy. Also, the use of liposomes, microcapsules or microspheres, inclusion complexes, or other types of carriers is contemplated.

Formulations for use with a metered dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the therapeutic agent, and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Nasal or other mucosal and systemic delivery of the therapeutic agent is also contemplated. Nasal delivery allows the passage to the blood stream directly after administering the composition to the nose, without the necessity for deposition of the product in the lung. Formulations for nasal delivery include those with dextran or cyclodextran and saponin as an adjuvant.

Methods of Stimulating an Immune Response:

In a typical immunization regime employing the vaccines of the present invention, the formulations may be administered in several doses (e.g. 1-4). The dose will be determined by the immunological activity the composition produced and the condition of the patient, as well as the body weight or surface areas of the patient to be treated. The size of the dose also will be determined by the existence, nature, and extent of any adverse side effects that may accompany the administration of a particular composition in a particular patient. For example, if the compositions are cells comprising the vaccines, the number of cells will be calculated and/or the amount of gp96-Ig which is being secreted will be calculated prior to administration to a patient.

The compositions of the present invention may be administered via a non-mucosal and systemic or mucosal and systemic route. These administrations may include in vwvo administration via parenteral injection (e.g. intravenous, subcutaneous, and intramuscular) or other traditional direct routes, such as buccal/sublingual, rectal, oral, nasal, topical (such as transdermal and ophthalmic), vaginal, pulmonary, intraarterial, intraperitoneal, intraocular, or intranasal routes or directly into a specific tissue. Alternatively, the compositions of the invention may be administered by any of a variety of routes such as oral, topical, subcutaneous, mucosal and systemic, intravenous, intramuscular, intranasal, sublingual, transcutaneous, subdermal, intradermal and via suppository. Administration may be accomplished simply by direct administration using a patch, needle, catheter or related device, at a single time point or at multiple time points.

Immunization via the mucosal and systemic surfaces offers numerous potential advantages over other routes of immunization. The most obvious benefits are 1) mucosal and systemic immunization does not require needles or highly-trained personnel for administration, and 2) immune responses are raised at the site(s) of pathogen entry, as well as systemically (Isaka et al. 1999; Kozlowski et al. 1997; Mestecky et al. 1997; Wu et al. 1997).

Extended Release Systems:

A first extended release system includes matrix systems, in which the agent is embedded or dispersed in a matrix of another material that serves to retard the release of the agent into an aqueous environment (i.e., the luminal fluid of the GI tract). When the agent is dispersed in a matrix of this sort, release of the drug takes place principally from the surface of the matrix. Thus the drug is released from the surface of a device, which incorporates the matrix after it diffuses through the matrix or when the surface of the device erodes, exposing the drug. In some embodiments, both mechanisms can operate simultaneously. The matrix systems may be large, i.e., tablet sized (about 1 cm), or small (<0.3 cm). The system may be unitary (e.g., a bolus), may be divided by virtue of being composed of several sub-units (for example, several capsules which constitute a single dose) which are administered substantially simultaneously, or may comprise a plurality of particles, also denoted a multiparticulate. A multiparticulate can have numerous formulation applications. For example, a multiparticulate may be used as a powder for filling a capsule shell, or used per se for mixing with food to case the intake.

In a specific embodiment, a matrix multiparticulate, comprises a plurality of the agent-containing particles, each particle comprising the agent and/or an analogue thereof e.g. in the form of a solid solution/dispersion with one or more excipients selected to form a matrix capable of controlling the dissolution rate of the agent into an aqueous medium. The matrix materials useful for this embodiment are generally hydrophobic materials such as waxes, some cellulose derivatives, or other hydrophobic polymers. If needed, the matrix materials may optionally be formulated with hydrophobic materials, which can be used as binders or as enhancers. Matrix materials useful for the manufacture of these dosage forms such as: ethylcellulose, waxes such as paraffin, modified vegetable oils, carnauba wax, hydrogenated castor oil, beeswax, and the like, as well as synthetic polymers such as poly(vinyl chloride), poly(vinyl acetate), copolymers of vinyl acetate and ethylene, polystyrene, and the like. Water soluble or hydrophilic binders or release modifying agents which can optionally be formulated into the matrix include hydrophilic polymers such as hydroxypropyl cellulose (HPC), hydroxypropyl methyl cellulose (HPMC), methyl cellulose, poly (N-vinyl-2-pyrrolidinone) (PVP), poly(ethylene oxide) (PEO), poly(vinyl alcohol) (PVA), xanthan gum, carrageenan, and other such natural and synthetic materials. In addition, materials, which function as release-modifying agents include water-soluble materials such as sugars or salts. Preferred water-soluble materials include lactose, sucrose, glucose, and mannitol, as well as hydrophilic polymers like e.g. HPC, HPMC, and PVP.

In a specific embodiment, a multiparticulate product is defined as being processed by controlled agglomeration. In this case the agent is dissolved or partly dissolved in a suitable meltable carrier and sprayed on carrier particles comprising the matrix substance.

Dose:

An effective dose of a composition of the presently disclosed subject matter is administered to a subject in need thereof. A “therapeutically effective amount” or a “therapeutic amount” is an amount of a therapeutic composition sufficient to produce a measurable response (e.g., a biologically or clinically relevant response in a subject being treated). The response can be measured in many ways, as discussed above, e.g. cytokine profiles, cell types, cell surface molecules, etc. Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the therapeutic composition, the route of administration, combination with other drugs or treatments, the severity of the condition being treated, and the condition and prior medical history of the subject being treated. However, it is within the skill of the art to start doses of the compound at levels lower than required to achieve the desired therapeutic effect and to gradually increase the dosage until the desired effect is achieved. The potency of a composition can vary, and therefore a “treatment effective amount” can vary. However, using the assay methods described herein, one skilled in the art can readily assess the potency and efficacy of a candidate compound of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention. Embodiments of inventive compositions and methods are illustrated in the following examples.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

### Example 1

### SIV Vaccine Expressing Gp96-Ig, SIV Retanef and Gag-Pol-Env Antigens Induces Poly-Specific Systemic and Mucosal and Systemic Immunity in Rhesus Macaques

Materials and Methods

293-SIV-gp96-Ig Vaccine Cells:

HEK-293 cell line was obtained from the American Tissue Culture Collection and transfected with the cDNAs encoding SIV env, gag, pol and retanef and gp96-Ig and expression verified by Western blots for the SIV genes and by ELISA for secreted gp96-Ig in the supernatant. Injection of cells secreting gp96 in vivo over a long period of time is many-fold more effective than injecting purified gp96. Therefore irradiated, transfected 293 cells that secrete 1, 10 or 50 mg gp96-Ig-SIV complexes in 24 h were injected.

Animals and Vaccination Schedule:

All animals used in this study were colony-bred rhesus macaques (Macaca mulatta) obtained from Covance Research Products (Alice, Tex.). The animals were housed and handled in accordance with the standards of the Association for the Assessment and Accreditation of Laboratory Animal Care International. The care and use of the animals were in compliance with all relevant institutional (NIH) guidelines.

A total of 8 naive female macaques that carry the MHC class I Mamu-A*01 molecule were enrolled and divided in four groups. Macaques were immunized at weeks 0, 4 and 25 with intraperitoneal inoculations of irradiated, 293 (control, 50×10⁶) or SIV-retanef env, gag-pol and gp96-Ig transfected 293 cells (1, 5 or 50 μg).

Isolation of Lymphocytes from Blood and Tissues:

Mononuclear cells from blood and LNs were isolated by density-gradient centrifugation on Ficoll and resuspended in RPMI 1640 medium containing 10% fetal bovine serum (R-10). Rectal and vaginal pinches were treated with 1 mM of Ultra Pure Dithiothreitol for 30 min followed by incubation in calcium/magnesium-free Hank's balanced salt solution. Following the removal of epithelium and intraepithelial lymphocytes, the tissues were incubated with collagenase D (400 U/ml; Boehringer Mannheim, Mannheim, Germany) and DNase (1 mg/ml; Invitrogen) for 2.5 h at 37° C. in Iscove's modified Dulbecco's medium supplemented with 10% fetal bovine serum and penicillin—streptomycin. The dissociated mononuclear cells were than placed over 42% Percoll and centrifuged at 800 g for 25 min at 4° C. Lamina propria lymphocytes were collected from the cell pellet.

Intracellular Cytokine Staining.

SIV-specific CD4⁺ and CD8⁺ T-cell responses were detected using pools of 15-meric peptides overlapping by 11 amino acids covering entire Gag, Env, and Pol proteins of SIV mac239. Cells (1×10⁶) in RPMI 1640 medium (containing 10% human serum and antibiotics) were incubated in the absence or the presence of a specific peptide pool at 1 μg/ml of each peptide for 1 h or in the presence of the super-antigen staphylococcal enterotoxin B (Sigma, St. Louis, Mo., US) at a 1 mg/ml final concentration as a positive control.

The co-stimulatory mAb CD28 and CD49d (0.5 μg/ml; BD Pharmingen, San Diego, Calif.) were added to all the samples to maximize the detection of T cells with higher activation thresholds. Positive control was treated with staphylococcal enterotoxin B Brefeldin A (GolgiPlug; BD Biosciences) at a final concentration of 10 μg/ml was added, and the cells were incubated for an additional 5 h. The cells were washed, stained for the surface antigens with anti-human CD8β antibodies (clone 2ST8.5H7; Beckman Coulter), permeabilized by incubation in Cytofix/Cytoperm solution (BD Biosciences), and stained with anti-human IL2-FITC (clone MQ1-17H2), and anti-IFNγ-APC monoclonal antibodies (clone B27; BD Pharmingen). The results were calculated as the total number of cytokine-positive cells with background subtracted.

Flow Cytometry: For the detection of SIV-specific CD8⁺ T cells, cells were labeled with anti-human CD8β-PE (clone 2ST8.5H7; Beckman Coulter), anti-human CD28-FITC (clone CD28.2; BD Pharmingen), anti-human CD95-PECy5 mAbs (clone DX2; BD Pharmingen), and with allophycocyanin (APC)-conjugated (Molecular Probes) Gag 181-189 CM9 (p11C) (CTPYDINQM)-Mamu-A*01 and Tat 8-35 SL8 (TTPESANL)-Mamu-A*01 tetrameric complexes (Beckman Coulter) for 30 min at room temperature. For phenotypic analysis of the lamina propria and intraepithelial lymphocytes subsets, cells were labeled simultaneously with the following combinations of anti-human antibodies: CD8β-PC5, CD103-FITC and tetrameric complexes (CM9 or SL8). Intracellular staining for granzyme B was performed using the Cytofix/cytoperm kit (BD Pharmingen) and anti-human granzyme B-PE (Caltag). A total of 100,000 events were collected in the lymphocyte region (R1) and analyzed with CellQuest software (BD Biosciences).

Results

Gp96-Ig mediated innate immunity activation and expansion of antigen specific CD8 CTL ensues independent of CD4 help. Gp96-Ig, chaperoning SIV peptides, is secreted from vaccine cells and binds to CD91 and TLR2 and TLR4 on DC at the local injection site. Gp96-binding to DC results in DC activation, independent of CD40-L and CD4 cells, upregulation of B7.1 and B7.2, secretion of IL-12 and recruitment and activation of NK cells. Gp96-Ig is endocytosed together with its chaperoned peptides by the endocytic CD91 receptor.

Replacement of KDEL of gp96 with IgG1-Fc and transfection of the cDNA into cells containing SIV antigens results in secretion of gp96-Ig-SIV peptide complexes. The chaperoned peptide is cross presented by MHC I of the activated DC and primes cognate CD8 cells with about 20 million fold higher efficiency than the corresponding protein not chaperoned by gp96. CD4 cells are not participating and are not activated in this pathway, which is active in the absence of CD4 cells and even lymph nodes. The activation of cytotoxic activity in NK and CD8 CTL by SIV-gp96-Ig is expected to function even in SIV infected macaques with low CD4 cells and non-functional lymph nodes. Lack of CD4 activation provides safety against the risk of viral spread even during innate activation and adaptive CD8 clonal expansion.

293-SIV-Gp96-Ig Immunization Induced SIV-Gag- and Tat-Specific CD8 T Cell Expansion in the Rectal Mucosa:

Eight Rhesus macaques were sorted into 4 groups and immunized with 293-SIV Retanef-gp96-Ig-SIV gag-SIV gp160 by the intra-peritoneal route. The number of cells injected were secreting the quantity of product as indicated within 24 h (by ELISA). Vaccine cells were administered 3 times at 0, 4 and 25 weeks, and two animals received 293 alone (control). 5 days after every immunization, samples were harvested from the rectal mucosa. SIV-Gag- and Tat-specific CD8 T cells were detected by Mamu-A*01/Gag181-189 CM9 (CTPYDINQM; Gag_CM9) and Tat 28-35 SL8 (TTPESANL; Tat SL8) tetramer staining. In addition, cells were stained with monoclonal antibody to CD8β and analyzed by flow cytometry. After gating on the CD8β⁺ population, the percentage of tetramer-positive cells was determined at each time point.

293-SIV-Gp96-Ig Immunization Induced Antigen Specific CD8 LPL in Jejunum, Ileum and Colon:

5 days after a 3rd immunization, LPL were harvested from the monkeys that had been vaccinated with 293 (control) or with 1, 5 or 50 mg of 293-SIV Retanef-gp96-Ig-SIV gag-SIV gp160. SIV-Gag-specific CD8 T cells were detected by Mamu-A*01/Gag181-189 CM9 (CTPYDINQM; Gag_CM9) and SIV-Tat-specific CD8 T cells by Mamu-A*01/Tat 28-35 SL8 (TTPESANL; Tat SL8) tetramer staining and CD8β and analyzed by flow cytometry.

293-SIV-gp96-Ig Immunization Induced SIV-Gag- and Tat-Specific CD8 T Cell Expansion in Peripheral Blood:

Eight Rhesus macaques were sorted into 4 groups and immunized with 293-SIV Retanef-gp96-Ig-SIV gag-SIV gp160 by the intra-peritoneal route. The number of cells injected were secreting the quantity of product as indicated within 24 h (by ELISA). Vaccine cells were administered 3 times at 0, 4 and 25 weeks, and two animals received 293 alone (control). 5 days after every immunization, samples were harvested from the rectal mucosa. SIV-Gag- and Tat-specific CD8 T cells were detected by Mamu-A*01/Gag181-189 CM9 (CTPYDINQM; Gag_CM9) and Tat 28-35 SL8 (TTPESANL; Tat SL8) tetramer staining. In addition, cells were stained with monoclonal antibody to CD8b and analyzed by flow cytometry. After gating on the CD8β⁺ population, the percentage of tetramer-positive cells was determined at each time point.

293-SIV-Gp96-Ig Immunization Induced SIV-Gag- and Tat-Specific CD8 T Cell Expansion in the Intra-Epithelial Compartment:

Eight Rhesus macaques were sorted into 4 groups and immunized with 293-SIV Retanef-gp96-Ig-SIV gag-SIV gp160 by the intra-peritoneal route. The number of cells injected were secreting the quantity of product as indicated within 24 h (by ELISA). Vaccine cells were administered 3 times at 0, 4 and 25 weeks, and two animals received 293 alone (control). 5 days after every immunization, samples were harvested from the rectal mucosa. SIV-Gag- and Tat-specific CD8 T cells were detected by Mamu-A*01/Gag181-189 CM9 (CTPYDINQM; Gag_CM9) and Tat 28-35 SL8 (TTPESANL; Tat SL8) tetramer staining. In addition, cells were stained with monoclonal antibody to CD8β and analyzed by flow cytometry. After gating on the CD8β⁺ population, the percentage of tetramer-positive cells was determined at each time point.

Induction of Poly-Specific Immune Response after 293-SI-Gp96-Ig Immunization:

Induction of polyspecific immune response after 293-SIV-gp96-Ig immunization. Lamina propria lymphocytes (LPL) (A and B) and intraepithelial lymphocytes (IEL) form rectal mucosa (C) obtained from monkeys that have been vaccinated with 1, 5, 50 μg of 293-SIV-gp96-Ig or with 293 cells only (control) 5 days after 3rd vaccination. LPL were stimulated with anti-CD28 and anti-CD49d in the medium or in the presence of the specific peptide pools of 15-meric peptides overlapping by 11 amino acids covering entire Gag, Env and Tat proteins of SIVmac239. Cells were incubated for 6 h in the presence of Brefeldin A and stained for surface markers CD8β and intacellular cytokines: IFNγ-APC and IL-2-FITC. A minimum of 10 000 CD4 or CD8 cells was acquired in the gate.

Summary:

These results indicate that cell secreted gp96-Ig induces both, mucosal and systemic and systemic CD8-CTL based immunity. Gp96-Ig induced migration of effector memory cells equipped with cytotoxic molecules (granzyme B) into the mucosal and systemic compartment by inducing selective expression of gut-homing markers (CD103). The mucosal and systemic immune response to SIV-gp96-Ig vaccine in the LPL and IEL compartments was poly-specific and multifunctional. Thus, the cell secreted SIV/HIV-gp96 vaccines have great prophylactic and therapeutic potential.

### Example 2

### Powerful Mucosal and Systemic Immune Response in Rhesus Macaques (Macaca mulatta) in Response to Gp96-Ig-SIV Immunization

Materials and Methods

Plasmids Construction and DNA Transfection:

The retanef (rev-tat-nef, rtn) fusion-construct comprised the sequences of nef, rev, and tat genes which were derived from the published sequences of SIVmac 239 and SIVmac251 isolates. Retanef construct was cloned into an expression vector derived from the kanamycin-expressing pVR1332 (Vical Inc., San Diego, Calif.).

Retanef was inserted into one expression cassette of the bovine papilloma virus derived vector, B45 vector. The second expression cassette of the B45 vector already contained the gp96-Ig fusion construct (B45-gp96-Ig). The B45 vector has been approved for human use by the FDA and the OBA as well as the local IRB and IBC in vaccine studies for the treatment of patients with lung tumors. B45 is a bovine papilloma virus derived vector from which the potentially transforming genes ES, E6, and E7 have been removed as well as the L1 and L2 viral capsid genes. The vector has in addition to the neomycin and ampicillin resistance genes two expression cassettes, one for expression of gp96-Ig and the other for retanef fusion proteins. The B45 vector replicates as multi-copy episome and provides high levels of expression.

The plasmids pCMV-SIV-gp160 and pCMV-SIV-gag backbone of both plasmid vectors was derived from the kanamycin-expressing pVR1332 (Vical, San Diego, Calif.). The SIV gag expression vector contained the CMV promoter (without introns), the RNA-optimized SIV p57 gag coding region, and the bovine growth hormone polyadenylation site. To optimize for RNA expression, the previously identified gag inhibitory sequences (INS) were mutated by introducing multiple silent point mutations not affecting the encoded protein precursor, as previously described for HIV-1 gag. The RNA-optimized SIV gp160 env gene contains 29 point mutations eliminating the Rev-responsive elements and is conjugated at the 3′ untranslated region to the constitutive transport element of simian retrovirus type 1, which further promotes mRNA export. DNA plasmid preparations of a clinical-grade quality were produced by Qiagen (Hilden, Germany). Both plasmids were linearized prior to transfection. 293 cells were co-transfected with all 3 plasmids simultaneously and selected with G418. Transfections were performed using Effectene (QIAGEN, Valencia, Calif.) following the manufacturers' protocols.

293-gp96-Ig-SIV Vaccine Cells:

Human embryonic kidney (HEK)-293 cell line was obtained from the American Tissue Culture Collection (ATCC) and were grown in IMDM medium supplemented with 10% FCS, free of contamination by Micoplasma. Gp96-Ig-SIV vaccine cells were generated by transfection of 293 cells with plasmids encoding gp96-Ig and SIV rev-tat-nef (rtn) (B45-gp96-Ig-rtn), gag (pCMV-SIVgag), and gp160 (pCMV-SIVgp 160). Transfected cells were selected with 1 mg/ml of G418 (Sigma, St. Louis, Mo.). Protein expression was verified by Western blots and by ELISA for secreted gp96-Ig in the supernatant. Cells were irradiated with 120 Gy in a Co irradiator and stored frozen in Cryopreservation buffer (containing 10% Dimethyl Sulfoxide (DMSO), 0.9% Sodium Chloride, 0.5% Rhesus serum, 8.4% Sodium Bicarbonate) in aliquots of 10⁶, 5×10⁶ or 50×10⁶ cells until use. In tissue culture, the irradiated cells were unable to form colonies, indicating their inability to replicate. The cells were thus considered safe for use as vaccine cells.

Western Blot and ELISA:

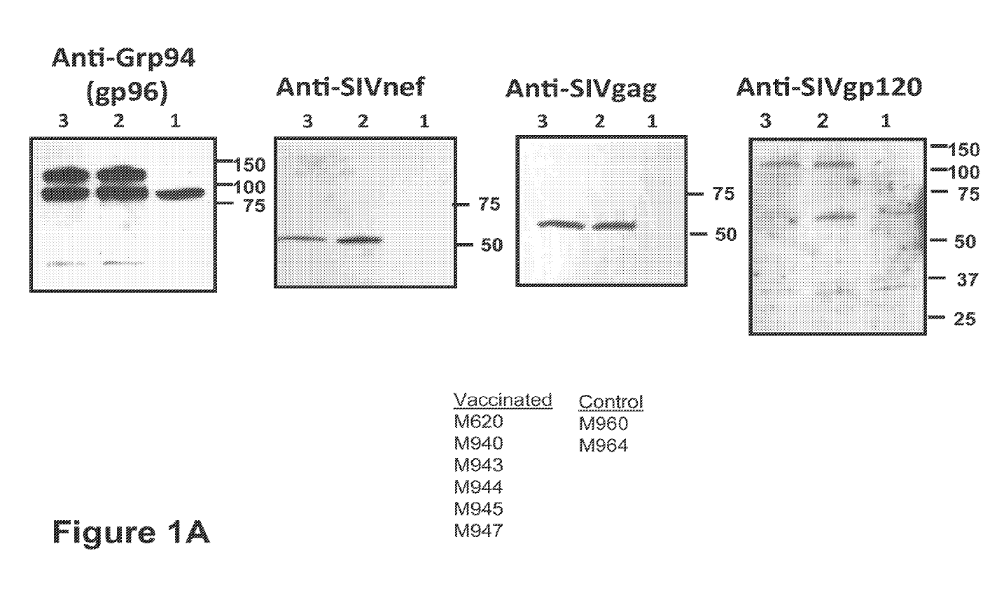

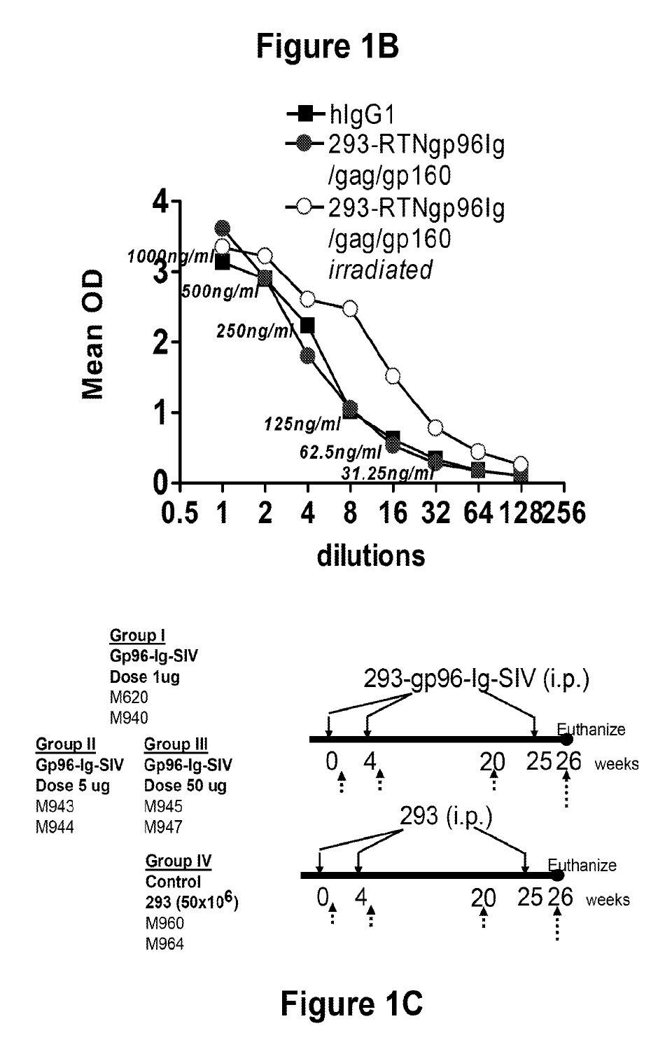

Total cell lysates were extracted with 1% NP-40 RIPA buffer with 50 mM Tris-HCl (7.5) and 150 mM NaCl in the presence of 20 μg/ml leupeptin, 1 mM 4-(2-aminoethyl)benzensulfonyl fluoride hydrochloride (AEBSF), 1 mM Na₃VO₄, and 20 g/ml aprotinin, and insoluble materials were removed by centrifugation. The concentration of proteins was determined by the Micro BCA protein assay reagent kit (Pierce, Rockford, Ill.). The sample suspension was boiled in the presence of 1% β-mercaptoethanol and 50 μg of total protein was loaded on a gel for electrophoresis and then transferred to a membrane (Millipore, Bedford, Mass.), for detection. Blocking of non-specific binding is achieved by 1% non-fat dry milk. After washing with PBS-Tween (0.05%), the blot was probed with different monoclonal antibodies: rat anti-Grp94 at 1:1000 for 1 h, mouse anti-SIVmac251Gag at 1:1000 dilution and mouse-anti-SIVmac251 Nef at 1:1000 dilution for 2 h, mouse anti-SIVmac251gp160/120 at 1:1000 dilution for 1 h. Blots were washed three times, and then incubated with peroxidase-conjugated goat anti-rat (for gp96) or goat anti-mouse IgG antibody (for gag, retanef and gp120) (Jackson ImmunoResearch Laboratories Inc., West Grove, Pa.) at a 1:10,000 dilution for 1 h. Gp96 and SIV antigens were visualized by an enhanced chemiluminescence detection system (Amersham Biosciences, Piscataway, N.J.) (FIG. 1A). 1×10⁶ cells were plated in 1 ml for 24 h and gp96-Ig production was determined by ELISA using anti-human IgG antibody for detection and human IgG1 as a standard (FIG. 1B).

Animals and Vaccination Schedule:

All animals used in this study were colony-bred rhesus macaques (Macaca mulatta) obtained from Covance Research Products (Alice, Tex.). The animals were housed and handled in accordance with the standards of the Association for the Assessment and Accreditation of Laboratory Animal Care International. The care and use of the animals were in compliance with all relevant institutional (NIH) guidelines.

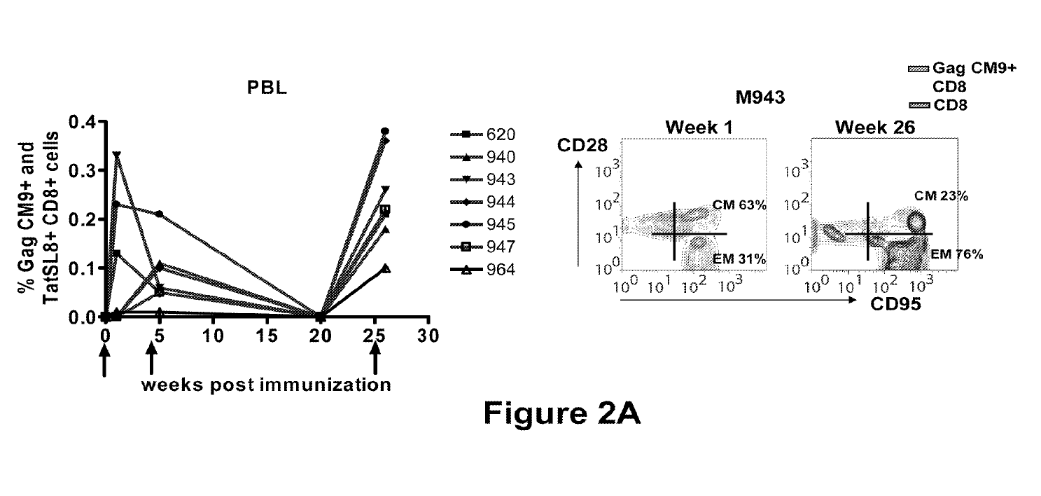

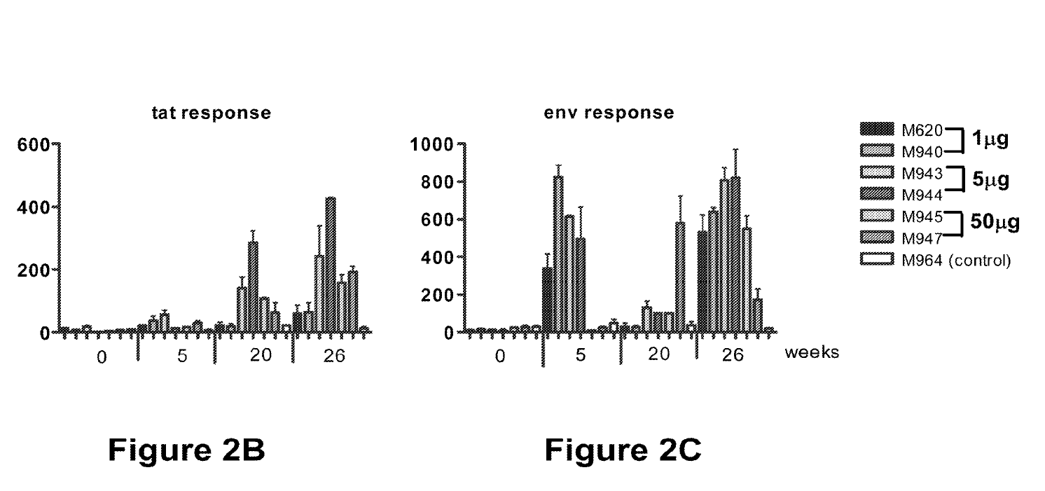

A total of 8 naïve females macaques, that carry the MHC class I Mamu-A*01 molecule were enrolled and divided in four groups, as indicated FIG. 2. The schedule of immunization shown in black arrows. Short, dashed arrows (5 days after each vaccination) represent time of the speciment collection (PBL, lymph nodes, BAL and rectal and vaginal pinches biopsies). Macaques in groups I, II and III were immunized at weeks 0, 4 and 25 with intraperitoneal inoculations of irradiated vaccine cells, 293-gp96-Ig-SIV that secrete 1, 5 or 50 μg of gp96-Ig at the rate of 106 cells/24 h. Irradiated 293 cells (50×10⁶) were injected in the animals from group IV (control). Macaque 964 initially enrolled in group IV, died during the study due to the post surgical complications.

Isolation of Lymphocytes from Blood and Tissues:

Mononuclear cells from blood and LNs were isolated by density-gradient centrifugation on Ficoll and resuspended in RPMI 1640 medium containing 10% fetal bovine serum (R-10). Rectal and vaginal pinches were treated with 1 m M of Ultra Pure Dithiothreitol for 30 min followed by incubation in calcium/magnesium-free Hank's balanced salt solution. Following the removal of epithelium and intraepithelial lymphocytes, the tissues were incubated with collagenase D (400 U/ml; Boehringer Mannheim, Mannheim, Germany) and DNase (1 mg/ml; Invitrogen) for 2.5 h at 37° C. in Iscove's modified Dulbecco's medium supplemented with 10% fetal bovine serum and penicillin—streptomycin. The dissociated mononuclear cells were than placed over 42% Percoll and centrifuged at 800 g for 25 min at 4° C. Lamina propria lymphocytes were collected from the cell pellet.

Intracellular Cytokine Staining.

SIV-specific CD4⁺ and CD8⁺ T-cell responses were detected using pools of 15-meric peptides overlapping by 11 amino acids covering entire Gag, Env, and Pol proteins of SIV mac239. Cells (1×10⁶) in RPMI 1640 medium (containing 10% human serum and antibiotics) were incubated in the absence or the presence of a specific peptide pool at 1 μg/ml of each peptide for 1 h. The co-stimulatory mAb CD28 and CD49d (0.5 μg/ml; BD Pharmingen, San Diego, Calif.) were added to all the samples to maximize the detection of T cells with higher activation thresholds. Positive control was treated with staphylococcal enterotoxin B (Sigma, St. Louis, Mo., US) at a 1 μg/ml. Brefeldin A (GolgiPlug; BD Biosciences) at a final concentration of 10 μg/ml was added, and the cells were incubated for an additional 5 h. The cells were washed, stained for the surface antigens with anti-human CD8β Abs (clone 2ST8.5H7; Beckman Coulter), permeablized by incubation in Cytofix/Cytoperm solution (BD Biosciences), and stained with anti-human IL2-FITC (clone MQ1-17H12), and anti-human-IFN-γ-APC mAbs (clone B27; BD Pharmingen). The results were calculated as the total number of cytokine-positive cells with background subtracted.

Flow Cytometry.

For the detection of SIV-specific CD8⁺ T cells, cells were labeled with anti-human CD8β-PE (clone 2ST8.5H7; Beckman Coulter), anti-human CD28-FITC (clone CD282; BD Pharmingen), anti-human CD95-PECy5 mAbs (clone DX2; BD Pharmingen), and with allophycocyanin (APC)-conjugated (Molecular Probes) Gag 181-189 CM9 (p11C) (CTPYDINQM)-Mamu-A*01 and Tat 8-35 SL8 (TTPESANL)-Mamu-A*01 tetrameric complexes (Beckman Coulter) for 30 min at room temperature. For phenotypic analysis of the lamina propria and intraepithelial lymphocytes subsets, cells were labeled simultaneously with the following combinations of anti-human antibodies: CD8β-PC5, CD103-FITC and tetrameric complexes (CM9 or SL8). Intracellular staining for granzyme B was performed using the Cytofix/cytoperm kit (BD Pharmingen) and anti-human granzyme B-PE (Caltag).

For the analysis, the central memory and effector memory T cell populations were identified as CD28⁺CD95⁺ and CD28⁻CD95⁺, respectively. The CCR7 marker was not used to define memory cells, as CD28 and CD95 identify similar T cell populations. A total of 100,000 events were collected in the lymphocyte region (R1) and analyzed with CellQuest software (BD Biosciences) and FlowJo.

ELISPOT:

ELISPOT was performed using the macaque IFN-γ-specific ELISPOT kits (U-Cytech, Utrecht, The Netherlands) to detect the number of Gag-specific, IFN-γ-producing cells. Ninety-six-well, flat-bottom plates were coated with anti-IFN-γ m Ab MD-1 overnight at 4° C. and blocked with 2% bovine serum albumin in phosphate-buffered saline for 1-3 h at 37° C. Cells (1×10⁵/well) were loaded in triplicate in RPMI 1640 containing 5% human serum and specific peptides (2 g/ml) or concanavalin—A (5 μg/ml) as a positive control. The plates were incubated overnight at 37° C. in 5% CO₂ and developed according to the manufacturer's guidelines (U-Cytech).

Results

SIV Vaccines Expressing Gp96-Ig, Retanef, Gag-Env Antigens in HEK293 Cells and Study Design.

Gp96-SIV vaccine cells were generated by transfection of human embryonic kidney (HEK) 293 cells with plasmids encoding gp96-Ig and SIV antigens: retanef (Rev-Tat-Nef fusion protein), gag and gp160. G418 selected 293 cells expressed the secreted form of gp96, gp96-Ig, in addition to endogenous gp96 (higher molecular weight band, 125 kDa, FIG. 1A) and comparable amounts of all three SIV antigens: Retanef (55 kD), gag (55 kD) and env (120 kD) (FIG. 1A). Gp96-Ig was secreted from both, irradiated and non-irradiated cells at a rate of 1000 ng/24 h by 10⁶ cells (FIG. 1B). In tissue culture, irradiated gp96-Ig-transfected cells were unable to form colonies, indicating their inability to replicate, but they still secreted gp96-Ig.

To assess the effect of gp96-Ig-SIV vaccine on systemic and mucosal and systemic immunity, 8 female rhesus macaques were enrolled in the study (as described under the Methods, section on animals). The animals were divided into four groups (I-IV) of two animals each (FIG. 1C). Macaques in groups I, II and III were immunized at weeks 0, 4 and 25, with intraperitoneal inoculations of irradiated vaccine cells (293-gp96-Ig-SIV) that secrete 1, 5 or 50 μg of gp96-Ig at the rate of 10⁶ cells/24 h. Irradiated 293 cells (50×10⁶) were injected in the animals from group IV (control). Injection of cells secreting gp96 in vivo over a long period of time is many-fold more effective than injecting purified gp96.

Gp96-Ig-SIV Induces SIV Specific CD8 T Cell Response in Peripheral Blood and Lymph Nodes:

CD8 T-cell responses to two immunodominant epitopes, Gag CM9 and Tat SL8/TL8 were studied, using major histocompatibility complex (MHC) I tetrameric complexes in all six gp96-vaccinated macaques and two of 293-treated macaques that express Mamu A*01 molecule. To assess systemic SIV-specific T-cell responses, frequency of SIV-gag-specific (CM9) and SIV-tat-specific (SL8⁺) CD8 T cells was determined by tetramer staining of freshly isolated peripheral blood mononuclear cells (FIG. 2A). 5 days after the first vaccine administration, SIV-specific CD8 T cells were detected in the 3 out of 6 vaccinated macaques. The frequency of SIV-specific CD8 T cells was the highest in the macaques from group II (M943) and III (M945) and interestingly, a second boost, 4 weeks later, did not induce further expansion of tetramer-specific CD8 T cells. The complete contraction of CD8 response was observed at week 20. Third vaccination (at week 25) induced rapid expansion of Gag-CM9⁺ and Tat-SL8⁺ cells in all vaccinated macaques. As expected, expansion of SIV-specific CD8 T cells was not observed in the control-293-vaccinated group (FIG. 2A). In addition to measurement of SIV-gag and SIV-tat specific CD8 T cell response, the ability of blood mononuclear cells to produce IFN-γ following stimulation with the entire Gag, Tat and Env peptide pool by ELISPOT (FIG. 1B). These results demonstrate that Gp96-Ig-SIV vaccination induced IFN-γ responses to all three SIV antigens tested. ELISPOT for IFN-γ responses to SI env was highly induced after second vaccination (interestingly only in groups I and II).

The quality of the T-cell response and not just the strength of the response may be important for the control of HIV/SIV replication. Therefore the differentiation and functional characteristics of established SIV-specific memory T cells induced by gp96-SIV vaccination were studied. Based on the co-expression of CD28 and CD95, memory CD8⁺ T cells in rhesus macaques have been broadly designated as T_{C}M (CD95⁺CD28⁺), TEM (CD95⁺CD28⁻) and naïve (CD95^{low}CD28^{int}). The surface expression of CD28 and CD95 was analyzed to define the central memory (T_{CM}) to effector memory (T_{EM}) differentiation axis among rhesus macaque CD8 T cells. SIV-gag CM9⁺ CD8 T cells in the peripheral blood of monkey (M943) that received 5 μg of gp96-SIV vaccine (group III) showed 66% TCM and 33% TEM phenotype 5 days after the first vaccination (FIG. 2C). Notably, boosted response induced predominant TEM phenotype (75% TEM). These results indicate that gp96-Ig vaccine in the blood, can elicit both, T_{CM} and T_{EM}-based T cell immunity and importantly, maintained SIV-specific T cells that are skewed toward TEM differentiation.

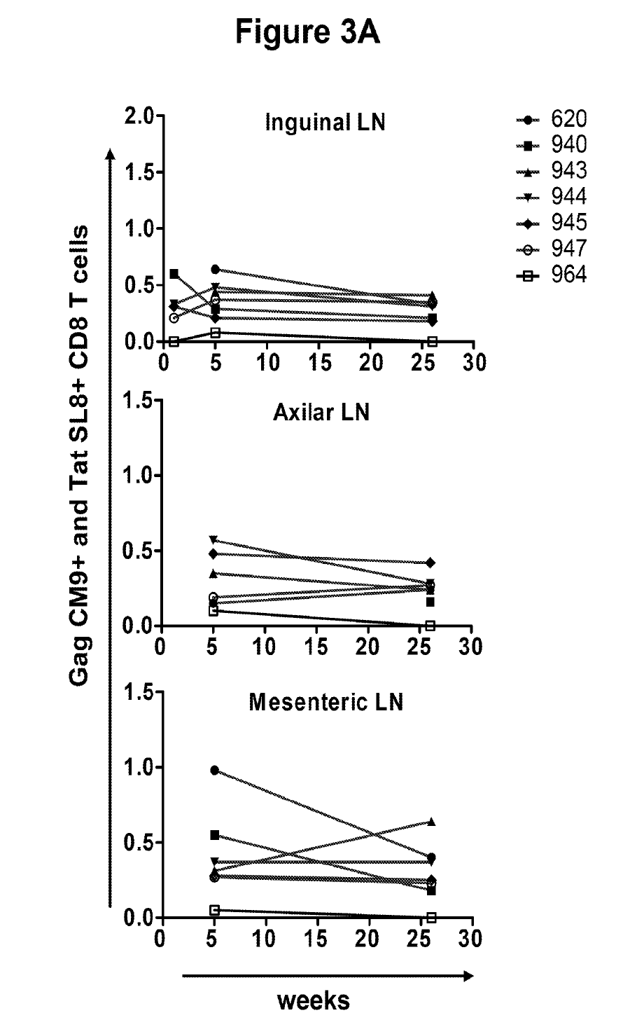

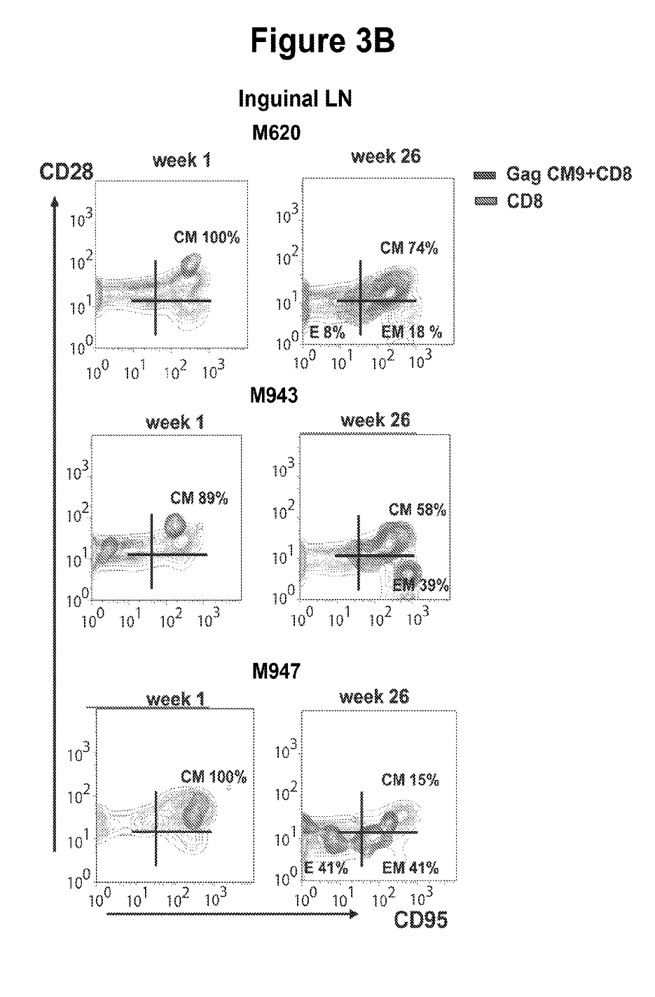

Gp96-Ig-SI vaccination was also associated with an increase in SIV-specific CD8 T cells in secondary lymphoid compartment, including: inguinal, axilar and mesenteric lymph nodes (FIG. 3A). Overall the frequency of SIV-specific CD8 T cells in all tested lymph nodes was not significantly changed during the course of immunization. The highest frequency of SIV-specific CD8 T cells was observed in the mesenteric lymph nodes from the monkey (M620) that received 1 μg of gp96-SIV vaccine (group I) (FIG. 3A). SIV-specific CD8 cell in lymph nodes of all vaccinated monkeys showed T_{CM} phenotype after the first vaccination (FIG. 3B). After the 3rd boost a decrease in the mean fluorescence intensity of CD28 molecule on the SIV-gag CM9⁺ CD8 T cells present in inguinal lymph nodes was observed. More pronounced difference in the differentiation pattern of established SIV-gag CM9⁺ CD8 T cells was observed in the inguinal lymph nodes from monkey M943 (FIG. 3B).

In summary these results indicate that gp96-SIV vaccine induces SIV-specific T cell response systemically. Importantly, elicited response in peripheral blood is highly skewed toward T_{EM} differentiation after boosting. SIV-specific memory response established in lymph nodes after primary immunization showed predominantly T_{CM} phenotype.

Predominant Gut Mucosal and Systemic Immune Response after Gp96-Ig-SIV Immunization:

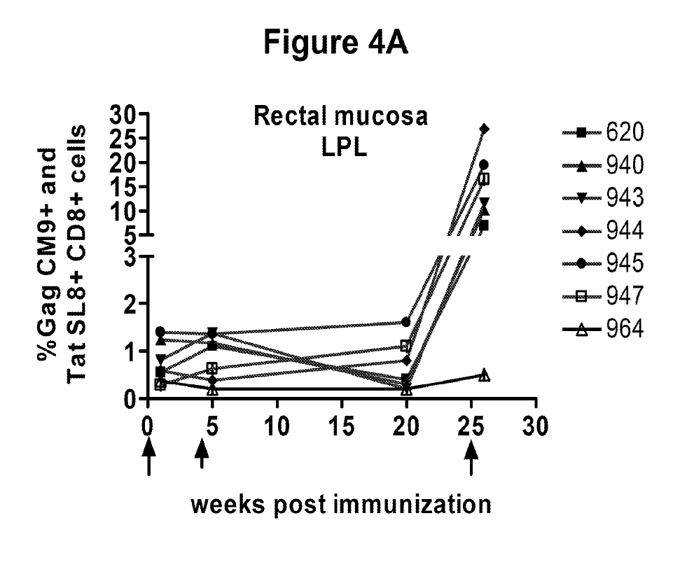

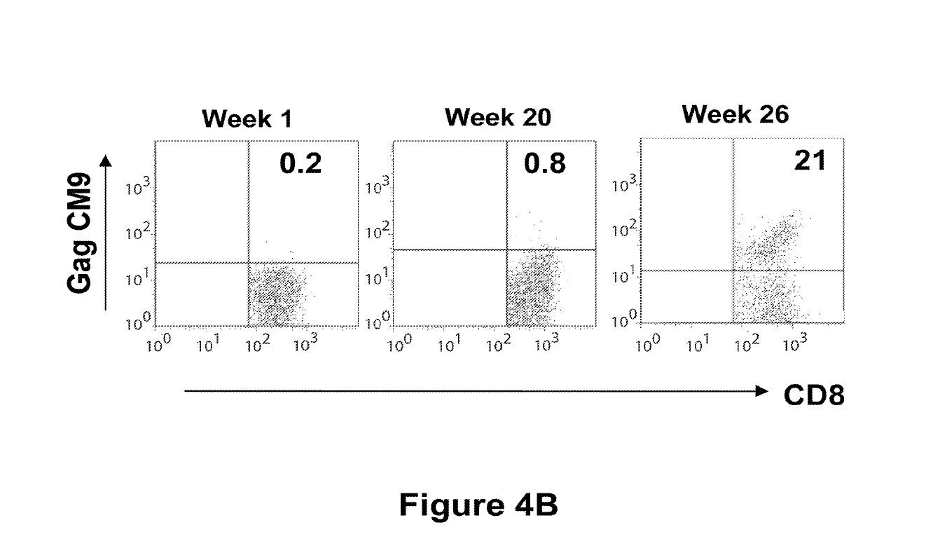

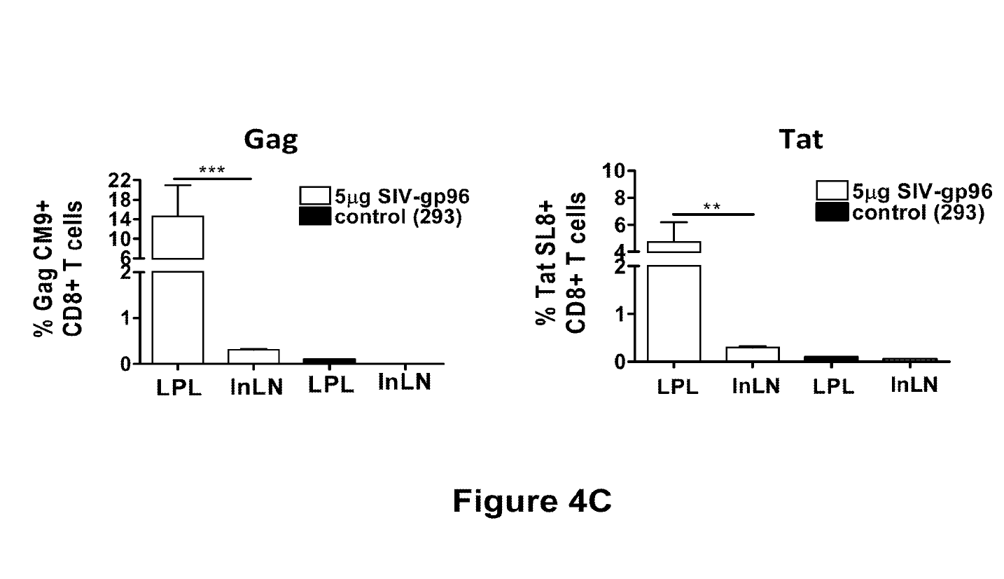

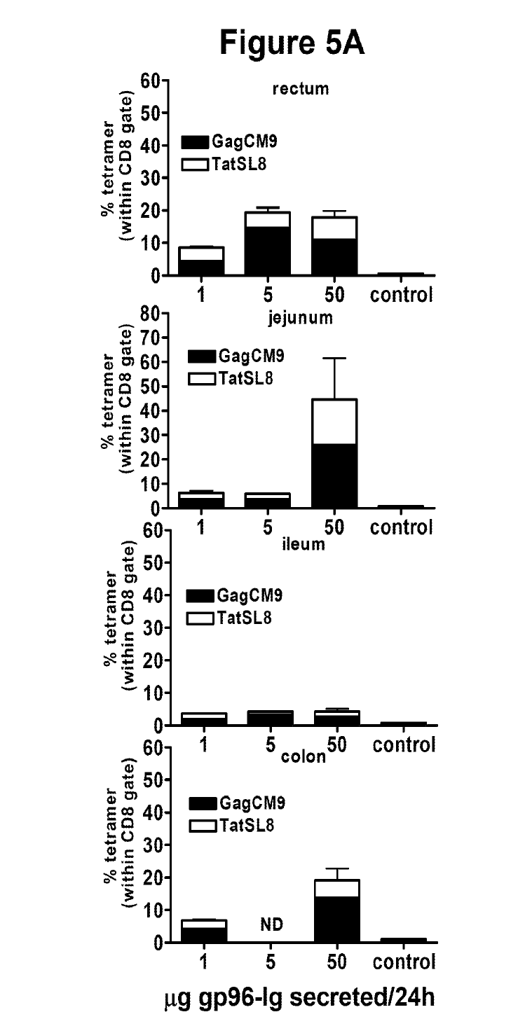

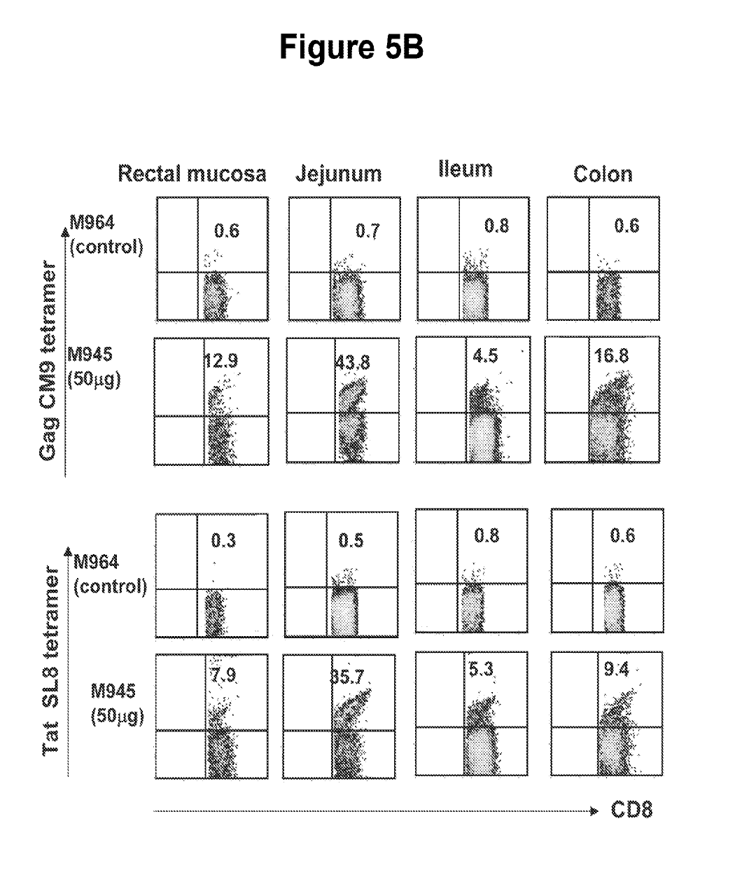

gp96-Ig immunization induces antigen specific effector memory CD8 T cells migrating to the intestinal mucosa. Thus, we hypothesized that gp96-SIV vaccine is capable of generating mucosal and systemic antigen-specific T_{EM}-type response. To assess SIV-specific T-cell responses in the intestinal tract, intraepithelial and lamina propria lymphocytes were isolated from rectal pinch biopsies by mechanical disruption and enzymatic digestion. Mononuclear cells were isolated on a Percoll gradient, resuspended in medium with 10% fetal calf serum and incubated overnight. Using this strategy 1-12×10⁶ lamina propria mononuclear cells were isolated from rectal tissue. Tetramer staining of the rectal lamina propria cell suspension demonstrated that gp96-SIV vaccine induced SIV-specific response in all 6 vaccinated macaques (FIGS. 4A, 4B). Gag-CM9 tetramer-specific CD8 T cells were detected already 5 days after first vaccination, at the frequency ranged from 0.59-1.2%. Vaccine boost, at week 4 did not induce significant increase in SIV-specific CD8⁺ T cells. The contraction of SIV-specific CD8 T cell response that was observed at week 20 in PBMC, was absent in rectal mucosa (FIG. 4A). Third vaccination (at week 25) resulted in dramatic expansion of Gag-CM9⁺ and Tat-SL8⁺ cells in all vaccinated macaques (p<0.001). In FIG. 4C, the frequency of SIV-gag⁺ and SIV-tat⁺ cells induced by SIV-gp96 vaccine, was examined in rectal lamina propria and in inguinal lymph nodes. Robust expansion of SIV-specific CD8 T cells was detected at the rectal mucosal and systemic tissue compared to inguinal lymph nodes. Difference in the frequency of tetramer⁺ cells within lamina propria and inguinal lymphocytes ranged from 8-fold in macaque M943 up to 22-fold in macaque M944 (FIG. 4C). These results clearly demonstrate that gp96 induces predominant mucosal and systemic immune response. Moreover, mucosal and systemic response to gp96 vaccine was observed not only at rectal mucosa, but also at jejunum, ileum and colon (FIGS. 5A, 5B). In all tested tissues (jejunum, ileum and colon) the frequency of SIV-specific CD8 T cells was the highest in the animals that received high vaccine dose (group III) (FIGS. 5A, 5B), except in the rectal tissue where the expansion of SIV-specific CD8 T cells in the animals from group II and III was comparable. Frequency of SIV-specific CD8 T cells at jejunum was dramatically increased in animal that received high vaccine dose (group III, M945) (FIGS. 5A, 5B). F or all vaccinated animals, the lowest frequency of SIV-specific CD8 T cells was observed in ileum.

Gp96-Ig-SIV Vaccines Induces SIV Specific Immune Response in the Gut Intraepithelial Compartment:

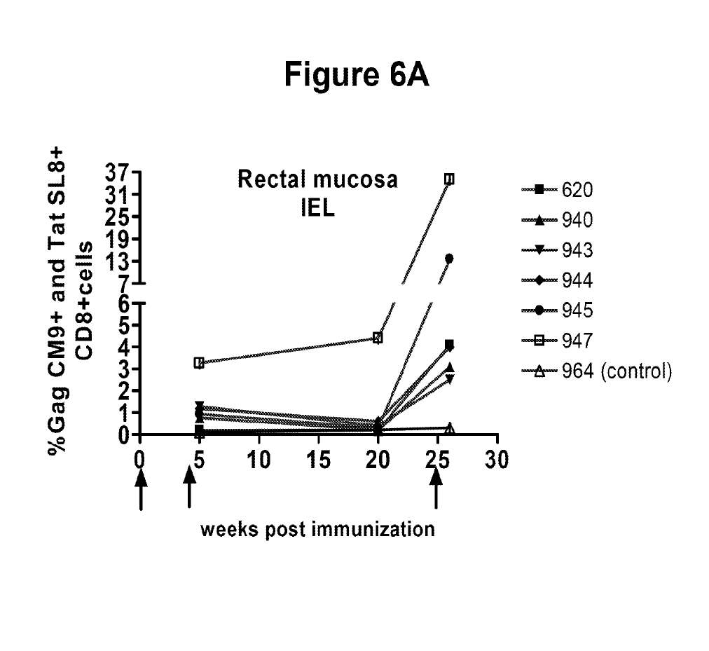

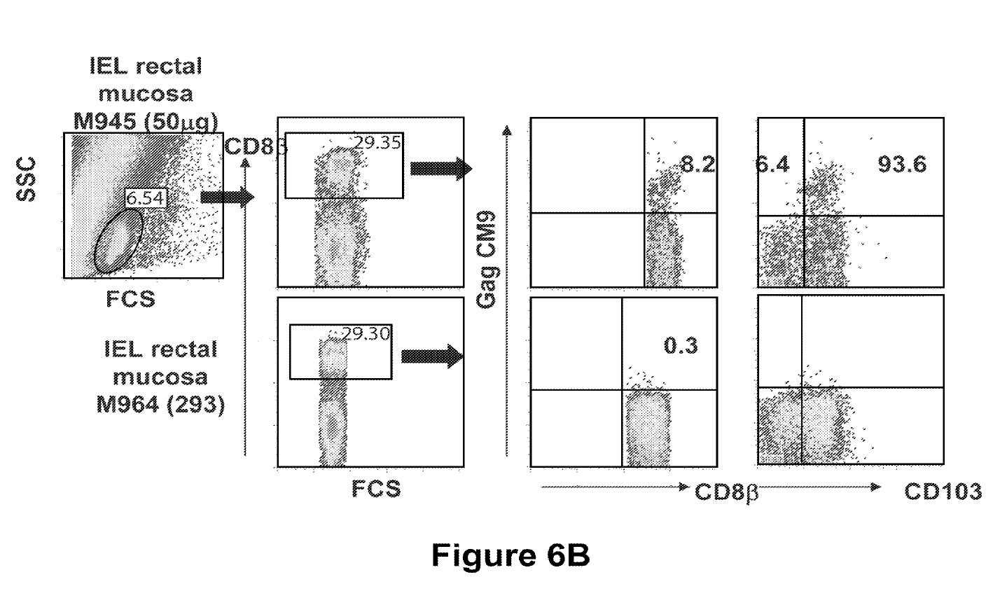

The presence of T cell response with cytolytic capacity at the portal of entry, is a desirable feature for combating an HIV/SIV infection. In order to explore in more details the effect of gp96-SIV vaccine on the induction of specific mucosal and systemic immunity, intraepithelial lymphocytes (IELs) and lamina propria lymphocytes (LPLs) were collected and analyzed separately. gp96 vaccine induced SIV-specific CD8 T cell response not only in lamina propria (FIGS. 4A,4B) but also in the intraepithelial compartment (FIG. 6A). Tetramer-binding CD8 T cells were detected in all vaccinated macaques 5 days after first vaccination, with the frequency rate comparable to the ones find in lamina propria (FIGS. 4A, 4B and 6A, 6B). At week 20 detectable level of Gag-CM9-tetramer⁺ CD8 T cells were found only in one macaque. In the lamina propria, in 3 out of 6 macaques contraction of SIV-specific response was observed at week 20. After the third vaccination, expansion of SIV-specific CD8 T cells was observed in all vaccinated animals (FIGS. 6A, 6B). Frequency of SIV-specific CD8 T cells was dramatically increased in the animals from group III (especially in the animal M947).

It was found that SIV-Gag-specific CD8 T cells induced by gp96-Ig-SIV vaccine exclusively express CD103 molecule on their surface (FIG. 6B), confirming that gp96 vaccination-induced up-regulation of gut homing molecules on antigen specific CD8 T cells.

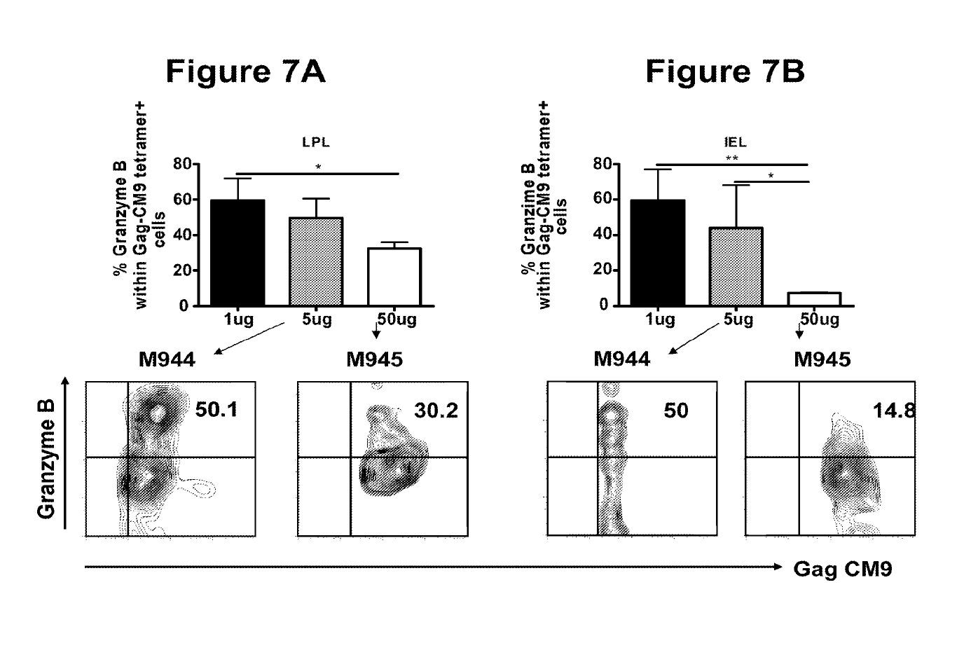

Without wishing to be bound by theory, it is thought that there is a direct role for resident mucosal and systemic effector CD8⁺ CTLs in the reducing initial viral burden and altering subsequent disease course following mucosal and systemic infection. To determine the effect of gp96-Ig-SIV vaccine on the cytotoxic potential of vaccine induced-mucosal and systemic SIV-specific immune response, experiments were conducted to ascertain the expression of cytotoxic molecule, granzyme B. s shown in FIGS. 7A, 7B, gp96-Ig-SIV-induced gag-specific CD8 T cells in the lamina propria and intraepithelial compartment, express granzyme B. Interestingly, frequency of granzyme B⁺ Gag-specific CD8 T positive cells, as well as mean fluorescent intensity (MFI) for granzyme B, was reverse to the vaccine dose received (monkeys with the lowest dose of gp96 vaccine showed the highest frequency of granzyme B+ Gag-specific CD8 T cells, p<0.005).

SIV-Specific Mucosal and Systemic Response is Poly-Specific:

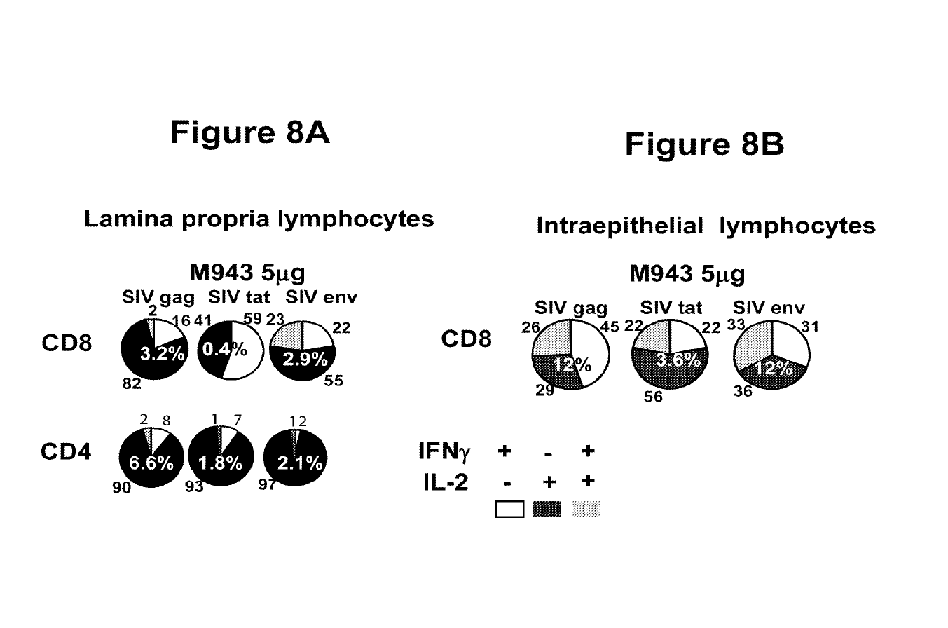

As discussed above, the quality of the SIV/HIV-1 CD8+ T cell response is more important than its magnitude. The poly-functional profile of gp96-SIV vaccine-induced immune response in the gut lamina propria and intraepithelial compartment was assessed by intracellular staining for IFN-γ and IL-2 after in vitro SIV-peptide stimulation. After the first vaccination, the frequency of SIV-specific IFN-γ-positive cells was low and they failed to co-express IL-2. However, five days after the third boost, an increase in the frequency of Gag-, Tat- and Env-specific IL-2 and IFN-γ-positive cells in all vaccinated macaques (only representative results for monkey 943 are shown) (FIGS. 8A, 8B). It was found that SIV-Gag response was predominant in CD4 as well as in CD8 compartment (3.2% and 6.6% of all CD4 or CD8 cells responding to Gag stimulation), followed by Env and Tat response. SIV-specific CD4 cells in lamina propria were almost exclusive producers of IL-2 (90° % of the total gag-specific CD4 T cells express IL-2), while SIV-specific CD8 cells were producing more IFN-γ (16% of total gag-specific CD8 T cells express IFN-γ and 82% IL-2). Differences in the quality of SIV-specific CD4 and CD8 response was more pronounced for Tat and Env-specific response: 93% of total tat- and 97% of total env-specific CD4 T cells expressed IL-2, while only 41% of tat- and 59% of env-specific CD8 T cells produced IL-2. Also, 23% of Env-specific CD8 T cells co-expressed both cytokines. Importantly, SIV-specific CD8 T cell response in the intraepithelial compartment was highly poly-specific: more than 25% of all gag-specific and 30% of all env-specific CD8 T cells co-express IL-2 and IFN-γ.

In summary, Gp96-SIV vaccine induced expansion of mucosal and systemic SIV-specific CD8 T cells, but more importantly, it also enhanced the functional quality of anti-SIV CD4 and CD8 T cells and resulted in the generation of poly-functional cells capable of co-producing IFN-γ and IL-2.

### Example 3

### Cell-Secreted Gp96-Ig-Peptide Complexes Induce Lamina Propria and Intraepithelial CD8⁺ Cytotoxic T Lymphocytes in the Intestinal Mucosa

The vaccine design developed as described below, uses the unique ability of the endoplasmic reticulum chaperone, heat shock protein gp96, also known as Grp94, to bind antigenic peptides and deliver them to antigen-presenting cells (APCs). To generate a secreted form of gp96, the endoplasmic reticulum retention sequence KDEL of gp96-cDNA was replaced with the IgG₁-Fc domain and hinge to generate the fusion protein gp96-Ig. Transfection of the cDNA of gp96-Ig into several cell lines (293, NIH-3T3, EG7, LLC), resulted in gp96 secretion by these cells, due to the lack of the endoplasmic reticulum retention sequence. Antigen specificity is provided by the peptide bound to gp96, which is taken up together with gp96 by APCs through the CD91 receptor. The repertoire of peptides bound by gp96 reflects the entire repertoire of peptides present inside the endoplasmic reticulum including those peptides imported by the TAP transporter. Only a small fraction of gp96-bound peptides, after further trimming, will be loaded onto major histocompatibility complex (MHC) class I. Peptide binding by gp96 is highly promiscuous, and the gp96 peptide repertoire is independent of the MHC molecules expressed by the cell. Therefore, isolated (cell-free) gp96 with its bound peptides, on uptake even by allogeneic DC, is ideally suited to cross-prime CD8-CTL responses against all kinds of intracellular antigens, including antigens derived from infectious agents and tumor antigens. Cell-released (cell-free) gp96 functions as potent internal adjuvant to signal traumatic/necrotic cell death and exerts it effect simultaneously as peptide carrier for antigen cross-presentation and cross-priming of antigen-specific CD8-CTL responses. Cell-free gp96 thus is a danger signal alerting DCs and other APCs through CD91, TLR2, TLR4, and SRA. Gp96 binding causes APC and DC activation and maturation and upregulation of B7, independent of CD40 signals: the adjuvant effect of gp96. Gp96 together with its associated peptides is engulfed through CD91 and the engulfed peptides are cross-presented by MHC class I of the engulfing APC and prime cognate CD8 cells and generate memory CD8 cells: cross-priming.

The role of secreted gp96-Ig was further described herein in the induction of mucosal and systemic immunity. This is the first report to present evidence that gp96-Ig immunization induces antigen-specific effector memory CD8T cells migrating to the intestinal mucosa. Thus, gp96-Ig vaccination strategy could be extremely useful for improving protection against a range of mucosal and systemic pathogens.

Materials and Methods

Cell Lines:

EG7 were transfected with the bovine papilloma-derived vector pCMGHis containing gp96-Ig as described 16 or with vector alone as control. NIH-3T3 cells were obtained from the ATCC.

Animals:

C57Bl/6-CD45.2⁺ and C57Bl/6-CD45.1⁺ mice were obtained from Jackson Laboratories (Bar Harbor, Me.). CS7BL/6 OT-I mice express a transgenic TCR (vα2, vβ 5.1.2) specific for the H-2K b-restricted peptide (ova) amino acid 257-264 (SIINFEKL) derived from chicken OVA. Gfp mice were crossed to OT-I mice to generate gfp-OT-I in the animal facility at the University of Miami according to institutional guidelines. The progeny mice were screened by PCR for the expression of the ova-TCR gene and for gfp fluorescence. All mice were used at 6-12 weeks of age.

Purification and Adoptive Transfer of OT-I Cells:

Single-cell suspensions of splenocytes were obtained from gfp-OT-I mice and were depleted of red blood cells with ammonium chloride buffer. Gfp-OT-I cells were purified by positive selection using anti-CD8a magnetic microbeads (Miltenyi Biotec, Auburn, Calif.) according to the manufacturer's instructions. More than 95% of the purified cells were CD8⁺, as determined by flow cytometric analysis. After Vα2 and Vβ 5.1/2 expression on purified cells were quantified by flow cytometry, one million OT-I cells were adoptively transferred in C57Bl/6 mice in a volume of 0.03 ml PBS IV.

OT-I cells (CD45.2⁺, gfp negative) were labeled with CFSE according to the manufacturer instructions (Invitrogen, Paisley, UK) and 10⁶ cells were injected IV into C57Bl/6-CD45.1 recipient mice.

Immunization:

Two days after adoptive transfer of one million gfp-OT-I or CFSE-labeled OT-I, 4×10⁶ EG7-gp96-Ig or 2×10⁶ NIH-3T3-OVA-gp96-Ig cells or control EG7 or NIH-3T3-OVA cells were injected IP in a volume of 0.5 ml PBS into recipient mice. After 5 days, cells were isolated from SPL, PEC, draining MLNs and the small intestine, and were subjected to further analysis.

Isolation of Small Intestinal Lymphocytes:

Small intestinal lymphocytes were isolated as described previously (Laky, K., Lefrancois, L & Puddington, L. J. Immunol. 158, 1417-1427 (1997)). In brief; the small intestine was cut from 0.5 cm below the stomach (from duodenum) to 1 cm above the cecum. IELs were isolated as follows; the small intestine was removed, flushed with calcium-magnesium-free buffer (10×calcium/magnesium-free Hank's balanced salt solution (Gibco BRL), 10×HEPES bicarbonate buffer (Sigma-Aldrich, St Louis, Mo.), 5% fetal bovine serum (Gibco BRL) and H₂O), Peyer's patches were dissected, and the intestines were cut longitudinally and then into pieces (2-5 mm). Gut pieces were treated with 1.3 mm EDTA (Sigma-Aldrich) in calcium-magnesium-free/fetal bovine serum buffer (30 min at 37° C., shaking at 200 r.p.m.). To isolate LPL, gut pieces were then treated with 100 U ml⁻¹ collagenase VIII (Sigma-Aldrich) in 5% RPMI 1640, 2 mm MgCl₂, 2 mm CaCl₂ (60 min at 37° C., shaking at 200 r.p.m.). Lymphocytes were purified on a 44/67% Percoll gradient (General Electric Healthcare, Piscataway, N.J.; 800×g at 20° C. for 20 min).

Antibodies and Flow Cytometric Analysis:

Single-cell suspensions were stained with 7-amino-actinomycin D, anti-CD3, anti-CD45.2, anti-B220, anti-CD8α, anti-CD8β, anti-CD45.2, anti-TCRαβ, anti-TCRγδ, anti-CD62L, anti-CD44, ant-CD11b, anti-CD11c, anti-F4/80, anti-MHC class II (A-E), anti-CD40, anti-CD80, anti-CD86, anti-CD013 (αE37), anti-α4β7, anti-CCR9, and anti-CCR7 antibodies (directly conjugated to fluorescein isothiocyanate, phycoerythrin, PE-Cy7, PerCP, APC, Pacific Blue, Pacific Orange (BD Pharmingen, San Jose, Calif.; eBioscience San Diego, Calif.; or R & D Systems, Minneapolis, Minn.). Four-seven parameter flow cytometric analysis was performed using a FacsCalibur and LSR II (BD Immunocytometry Systems, San Jose, Calif.) with FlowJo (Three Star, Ashland, Oreg.) or DIVA (BD Biosciences, San Jose, Calif.) software.

Intracellular Cytokine (IFN-γ, IL-2, and IL-17) and Granzyme B Staining:

Gut lymphocytes were isolated as described above, stained for surface anti-CD8 and then fixed/permeablized, and intracellular staining for granzyme B was performed. To enhance intracellular cytokine protein accumulation (IFN-γ and IL-2), gut lymphocytes were cultured with 20 n m SIINFEKL peptide and 10 ng ml⁻¹ of Brefeldin A (Sigma-Aldrich) for 5 h. Before staining, all cells were treated with purified anti-mouse CD16/CD32 (Fc-γIII/II receptor; BD Pharmingen). Granzyme B staining was performed on freshly isolated gut lymphocytes. Intracellular staining for IFN-γ, IL-2, and Granzyme B was performed using the Cytofix/cytoperm kit in accordance with the manufacturer's directions (BD Pharmingen).

Gut lymphocytes were stimulated with 1 μg ionomycin, 50 ng ml⁻¹ phorbol 12-myristate 13-acetate (Sigma-Aldrich), and 2 μm monensin (eBioscience) for 5 h at 37° C. After surface staining for CD8 (BD Pharmingen), cells were fixed/permeabilized and intracellular staining for IL-17F (eBioscience) was performed. Samples were analyzed on an LSR II with DIVA software.

In Vitro Cell Cultures:

Purified CD8⁺ OT-I cells (CD8⁺ T-cell isolation kit; Miltenyi Biotec) were labeled with CFSE according to the manufacturer's instructions (Invitrogen). For CD103⁺ and CD103 purification, PEC cells from 3T3-OVA-gp96 or PBS-treated mice at day 5 were sorted using anti-CD11c, anti-CD11b, anti-MHC class II, anti-F4/80 and anti-CD103 on FACS Aria (BD Biosciences). Before staining, cells were preincubated with Mouse BD Fc Block (BD Biosciences). DC fractions were 95-98% pure. DCs were pulsed with 2 nm SIINFEKL peptide for 1 h at 37° C., washed extensively, and 10⁵ DCs were incubated with 2×10⁵ CFSE-labeled OT-I cells in the flat-bottom 96-well plates in 200 ml complete medium. The phenotype of responding OT-I cells was assessed by flow cytometry after 5 days.

Statistical Analysis:

Statistical analyses were performed using paired or unpaired two-tailed Student's t-test.

Results

Induction of Adaptive CD8+β⁺ TCRαβ⁺ Antigen-Specific CD8⁺ LPL and IEL by Secreted Gp96-Ig.

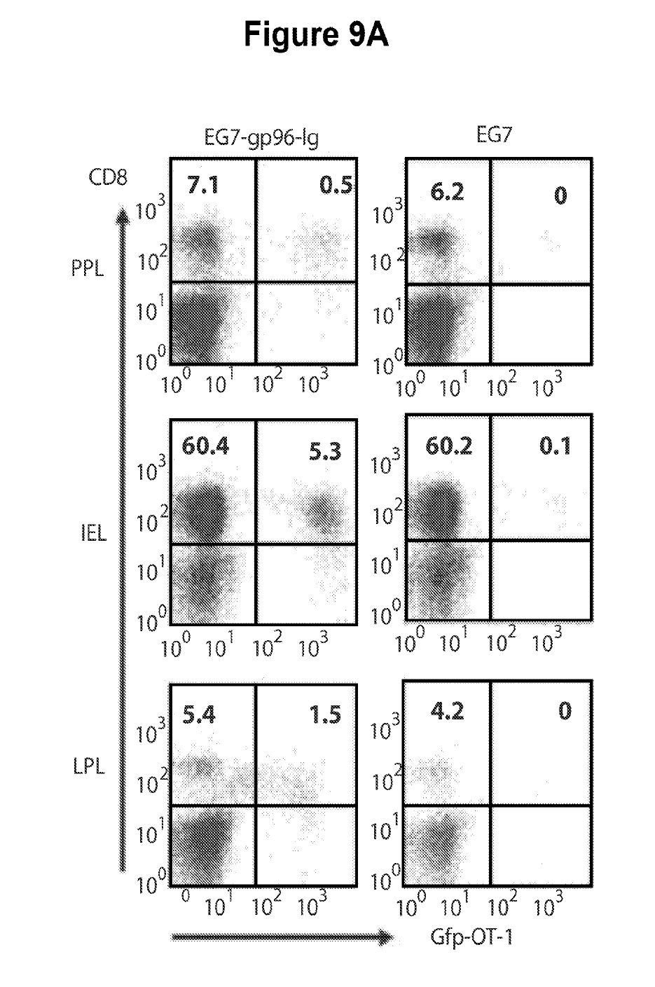

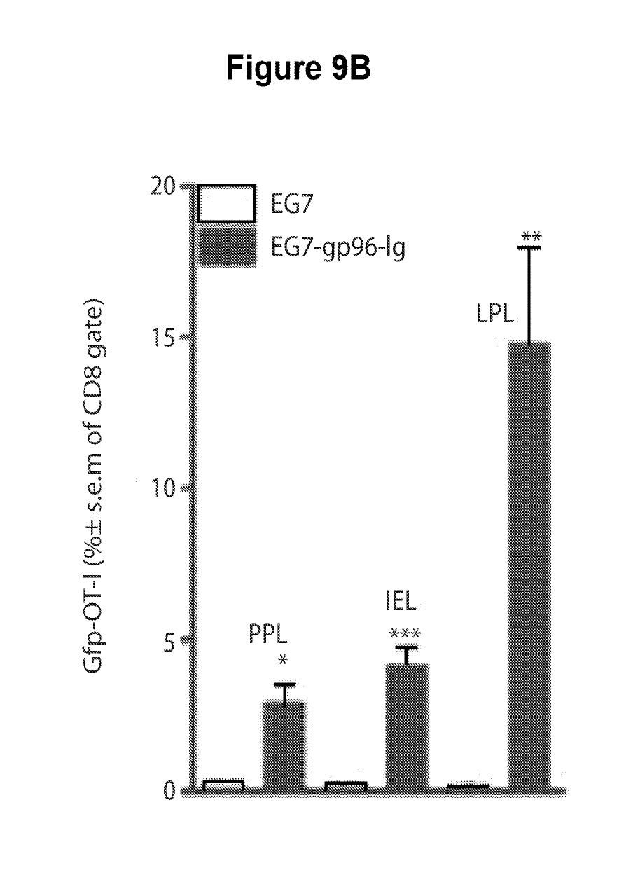

Secreted gp96-Ig peptide complexes provide within one molecular complex strong adjuvant properties to activate DC and antigen specificity for cross-presentation and cross-priming of CD8T cells. To determine whether gp96-Ig vaccination induced mucosal and systemic immunity, the EG7-gp96-Ig/OT-1 model was used. In this model the expansion of TCR transgenic, OVA-specific OT-I CD8T cells in response to EG7-gp96-Ig immunization is quantitated at several mucosal and systemic and peripheral sites. First, the phenotype of mucosal and systemic small intestine lymphocytes from non-immunized C57Bl/6 (B6) mice was characterized. Immunized B6 mice received IV, one million green fluorescent protein (gfp)-marked, TCR transgenic OT-I cells recognizing SIINFEKL (ova peptide) presented by K^{b}. After 2 days, mice were immunized IP with four million EG7-gp96-Ig or four million EG7 (derived from the EL4 lymphoma by OVA transfection). Four million EG7-gp96-Ig cells secrete 250 ng gp96-Ig within 24 h in culture. It was found that gp96-Ig secretion mediated robust CD8-CTL (OT-I) expansion reaching a peak on day 5; expansion was detectable locally in the peritoneal cavity (PEC) and systemically in draining LNs, blood, and spleen (SPL). In addition to the analysis of gfp-OT-I cells in the PEC and in the SPL, the frequency of gfp-OT-I was examined within Peyer's patch lymphocytes (PPLs), IELs, and LPL. EG7 cells (secreting OVA but not gp96) had virtually no effect on OT-I homing to mucosal and systemic sites, which remained close to zero frequency (FIGS. 9A and 9B). In contrast, a single IP immunization with four million EG7-gp96-Ig cells secreting 250 ng gp96-Ig in 24 h (in culture) caused considerable homing of OT-I to Peyer's patches (2.9±1.1% s.e.m. OT-I in the CD8 gate), to the intraepithelial compartment (4.1±1% s.e.m.) and to the LP (14.9±5% s.e.m.) (FIGS. 9A and 9B). Clearly, gp96-Ig IP vaccination is a powerful inducer of antigen-specific CD8 cells at mucosal and systemic sites.

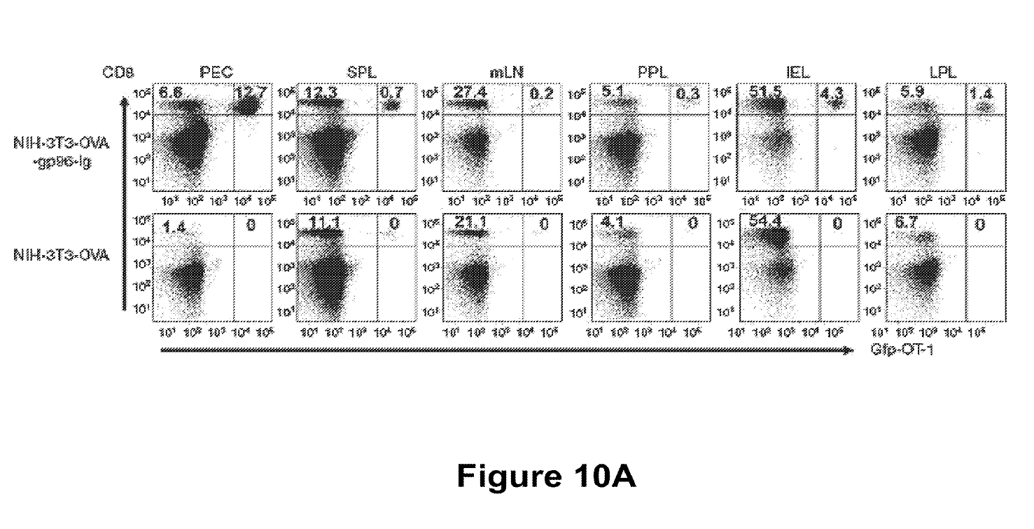

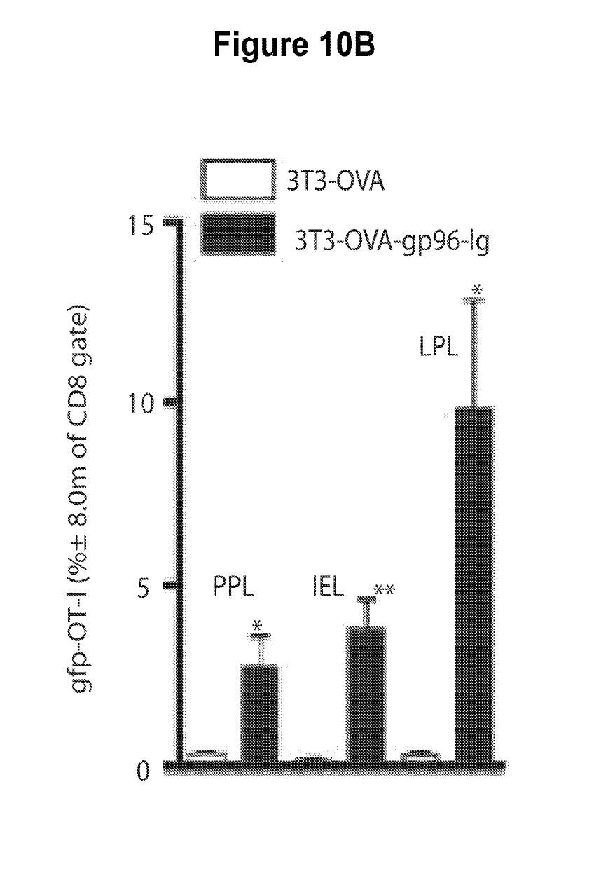

Since OT-I cells are K^{b} restricted, direct antigen presentation to OT-I by syngeneic EG7 cells is possible. To exclude direct antigen presentation, allogeneic NIH-3T3 cells transfected with OVA and gp96-Ig (3T3-OVA-gp96-Ig) were used, which can cause OT-I expansion only by MHC-class-1-mediated cross-presentation of OVA-derived SIINFEKL (FIGS. 10A, 10B). NIH-3T3-OVA cells, not transfected with gp96-18, were used as controls. IP vaccination with NIH-3T3-OVA-gp96-Ig induced OT-I homing to the mucosal and systemic compartments at a similar frequency as syngeneic EG7-gp96-Ig vaccination, namely 2.7±0.8, 4.1±0.8, and 9.8±3.1% s.e.m. in PPL, IEL, and LPL, respectively (FIGS. 9B and 10B). Very high frequencies of OT-1 CTL were also found at the site of the injection, the PEC (up to 60% of CD8 cells, ˜400,000 total OT-I), in the SPL (5%) and mesenteric LNs (1%). It is noteworthy that NIH-3T3-OVA cells, not secreting gp96-Ig, attract relatively few CD8 cells to the PEC, only 1.4% of the cells in the lymphocyte gate are CD8⁺ as opposed to almost 20% CD8 cells when gp96-Ig is secreted. This observation indicates that secreted gp96-Ig triggers immune responses that are targeted toward cytotoxic responses. OT-I expansion was not found in the animals that were injected with NIH-3T3-OVA (FIG. 10B) or NIH-3T3-gp96-Ig (not containing OVA), indicating that the antigen must be present in cell secreting gp96-Ig.

Gp96-Ig Immunization Induces Memory OT-I Cells that Express Receptors for Intestinal Homing and Cytotoxic Molecules.

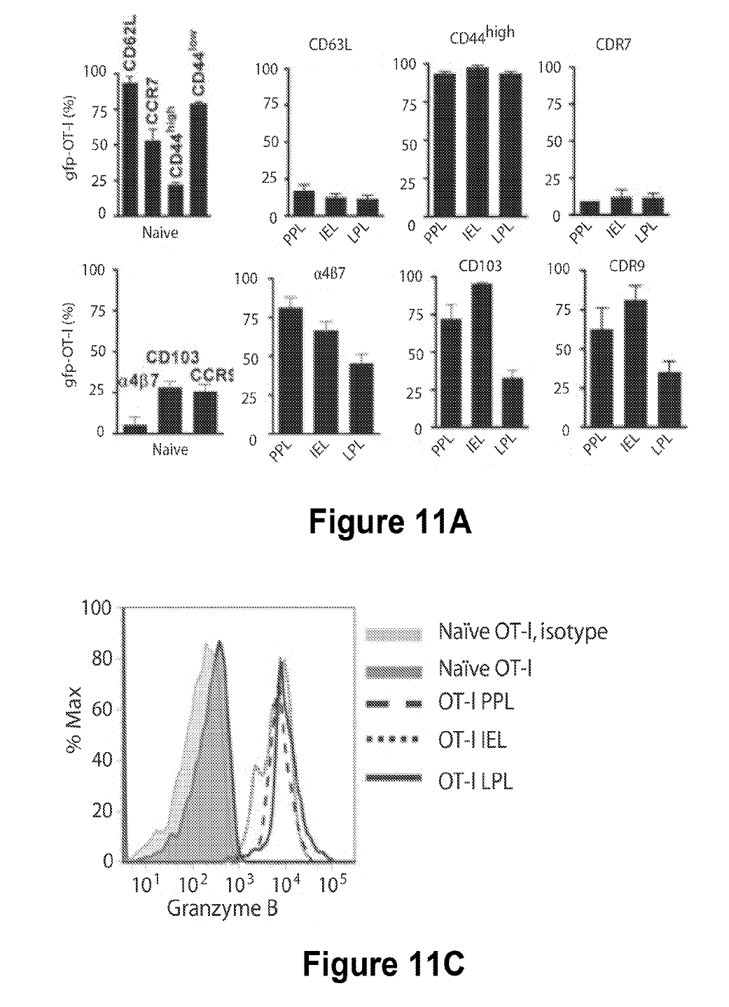

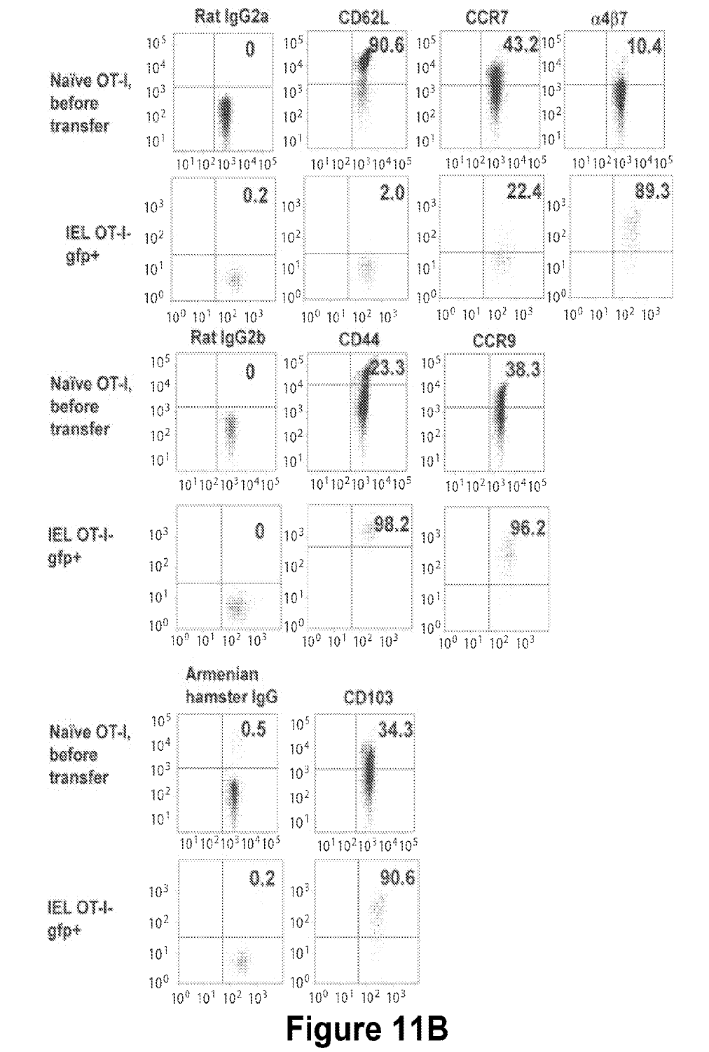

Gfp-OT-I cells isolated from mucosal and systemic compartments following gp96-Ig immunization are (CD44^{high} CD62^{low} CCR7^{low}; FIGS. 11A-11C) similar to the SPL and PEC data. It was found that mucosal and systemic OT-I cells retained their CD8 complex composition and, remained TCRαβ⁺ CD8αβ⁺ within all mucosal and systemic compartments. Cell-secreted gp96-Ig-peptide complexes however induced upregulation of the gut-homing molecules αEβ7 (CD103), α4β7, and CCR9 (FIGS. 11A-11C) in OT-I isolated from mucosal and systemic compartments.

Gp96-Ig vaccine also induced high levels of granzyme B in OT-I in all three gut-homing compartments, PPL, IEL, and LPL (FIGS. 11A-11C), indicating that gp96-Ig-induced, antigen-specific, adaptive CTL in mucosal and systemic compartments could contribute to the elimination of antigen-specific target cells and may serve as protection against mucosal and systemic virus infection.

Antigen-Specific Memory CD8T Cells that Migrated to the SPL or the Intestinal Mucosa after Gp96-Ig Immunization Differ in Phenotype and Function:

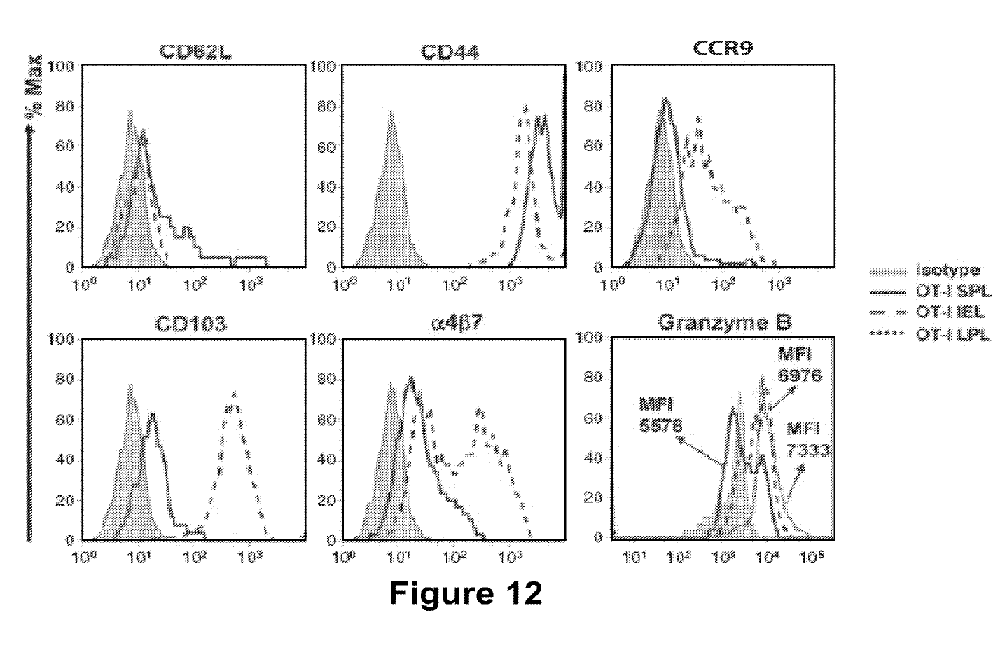

The memory phenotype may be coupled to anatomic location and that memory CD8T cells residing within the intestinal mucosa differ from their clonotypic counterparts within the SPL regarding phenotype, function, cell cycle, and cytokine receptor expression. Comparing the phenotype of gp96-Ig-induced SPL and IEL OT-I cells 5 days after gp96-Ig vaccination, CD62L^{low} CD44^{high} cells were found in both populations. However a subpopulation of CD62L^{high} cells was present only within SPL OT-I (FIG. 12). Further, in contrast to IEL OTI, SPL OT-I remained CD103^{low} CCR9^{low} and α4β7^{low} similar to naive OT-I. These data show for the first time that gp96-Ig induces expression of essential intestinal homing receptors on antigen-specific CTLs. Compared with immunizations with high doses of whole OVA, or Ova peptide with adjuvant, much lower doses of peptide bound to Gp96-Ig were required to induce OT-I activation and localization to the intestinal effector sites. The memory cells that accumulate in the intraepithelial compartment bear little resemblance to recirculating central or effector memory cells. Following gp96-Ig vaccination, only effector phenotypic OT-I cells migrated to the gut mucosa. LPL OT-I and IEL OT-I cells expressed the highest level of granzyme B with a mean fluorescent intensity (MFI) for granzyme B within IEL OT-I=6,976 and MFI LPL OT-I=7,333 compared with SPL MFI OT-I=5,576 (FIG. 12).

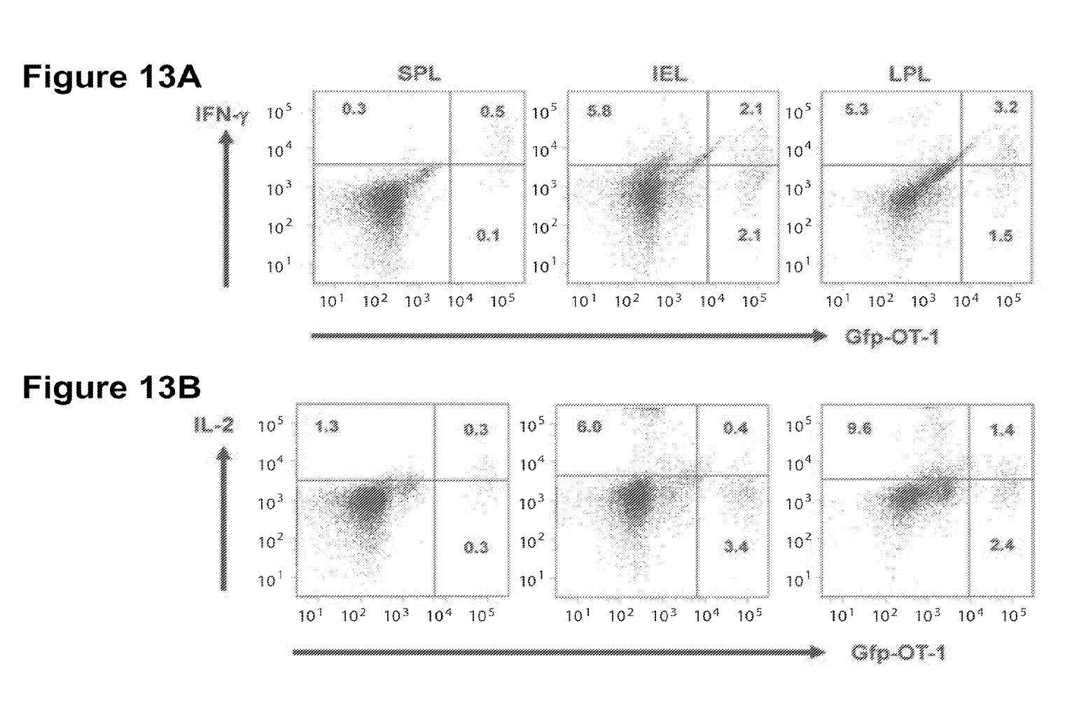

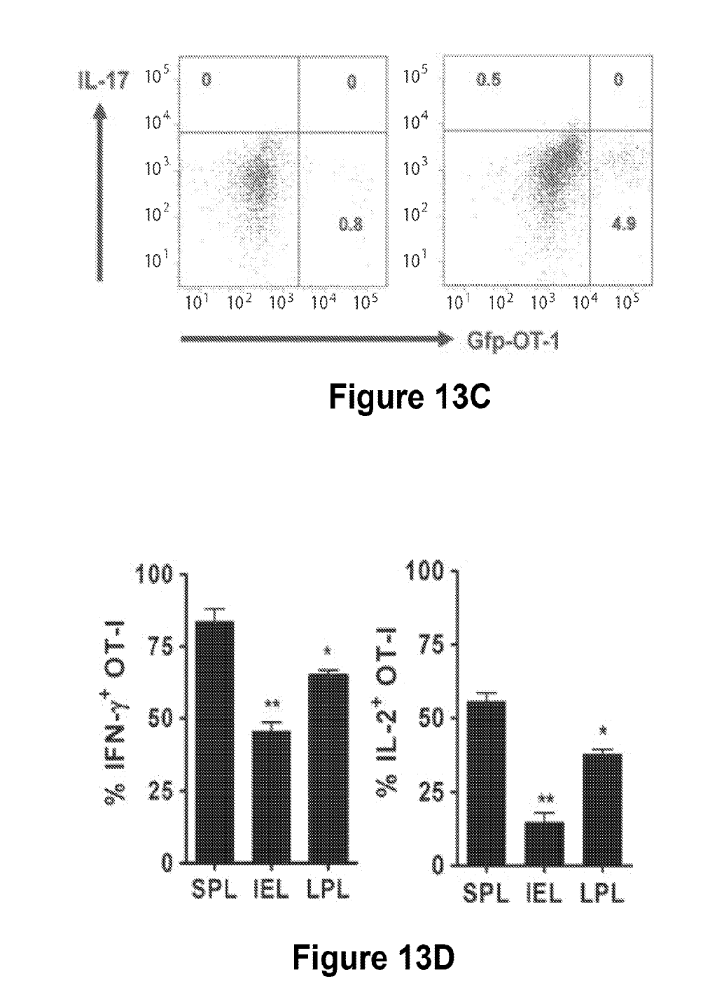

In contrast to memory SPL OT-I, relatively few IEL OT-I cells produced interleukin (IL)-2 on in vitro antigenic (SIINFEKL peptide) restimulation (FIGS. 13B and 13D). Most of the SPL OT-I produced proinflammatory interferon-γ (IFN-γ FIGS. 13A and 13D), whereas only half (50%) of IEL OT-I produced this cytokine (FIGS. 13A and 13D). Type 17 CD8⁺ T cells represent a response in defense against intracellular pathogens. However OT-I cells induced by gp96-Ig vaccination did not produce IL-17, systemically or in the mucosal and systemic compartment (FIG. 13C). The data show that gp96-Ig leads to the accumulation of highly cytotoxic antigen-specific CD8⁺ T cells within the mucosa with a reduced ability to produce inflammatory cytokines when compared to splenic OT-I.

Gp96-Ig Immunization Increases Frequency of CD103⁺ (αEβ7) DCs and Efficiently Induces CCR9 on Responding T Cells In Vitro.

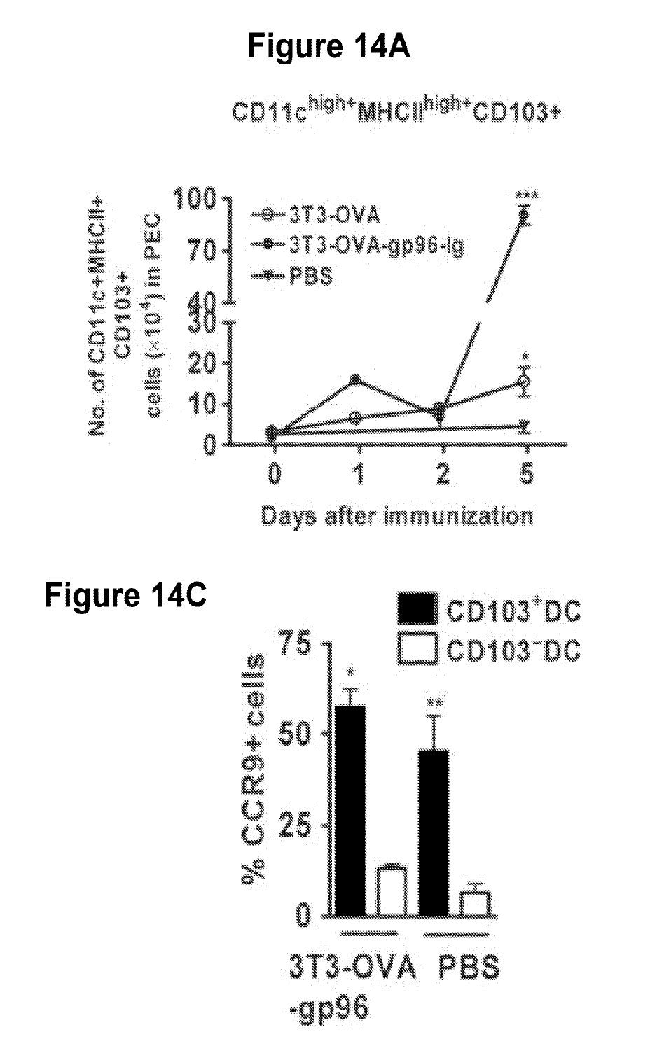

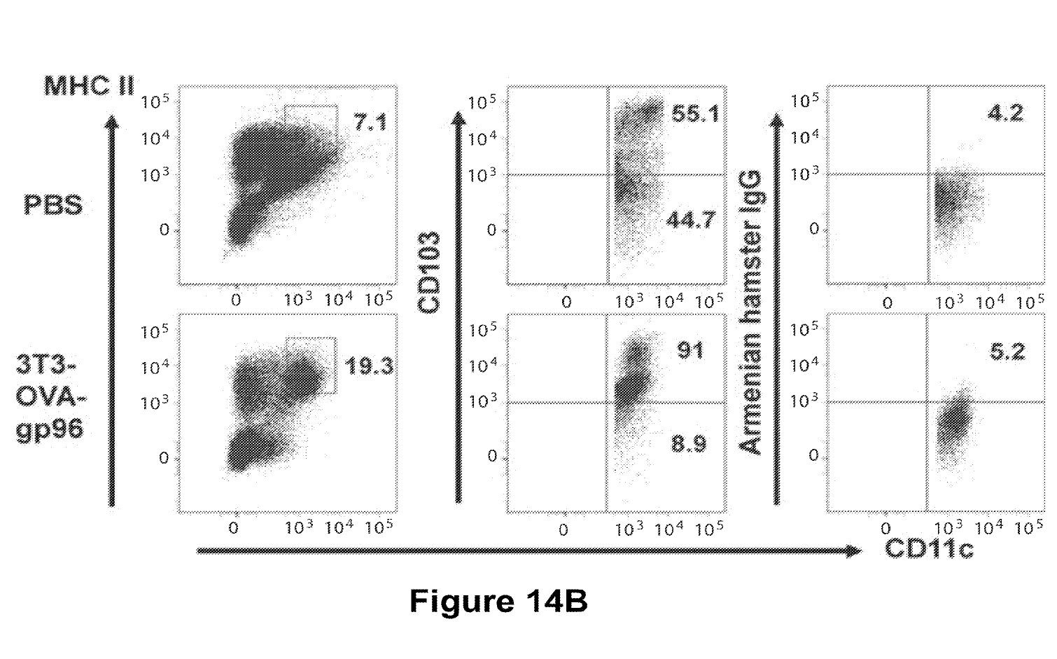

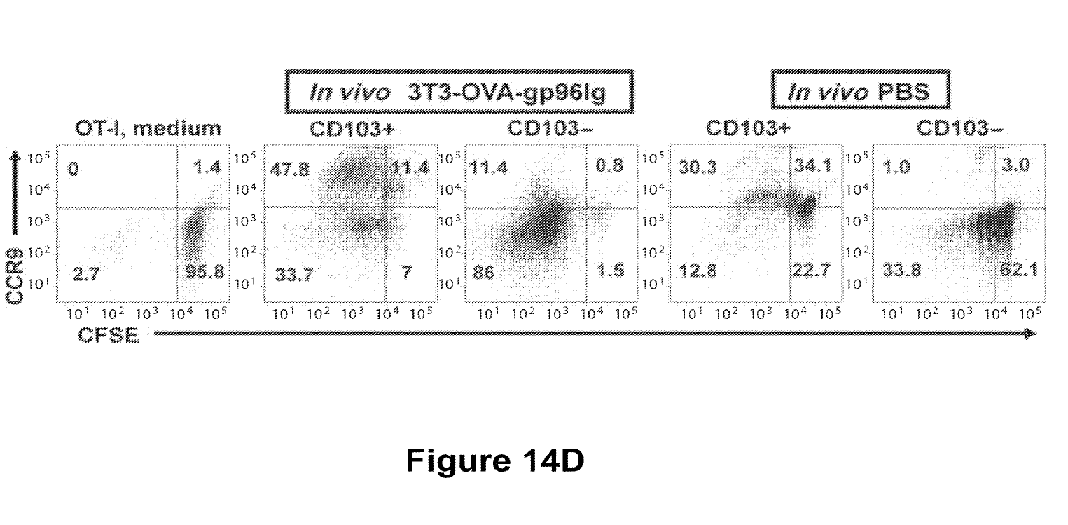

To activate CD8 and natural killer cells, secreted gp96-peptide complexes need to be taken up by DCs or peritoneal macrophages. CD103⁺ DCs are important for generation of gut-tropic CD8 effector cells as well as T regulatory cells. Also, CD103^{-/-} mesenteric lymph node (MLN) DCs are as efficient as wildtype MLN DCs at inducing CCR9. CD11c^{high} MHC class II^{high} cells from the PEC of control (phosphate-buffered saline, PBS) or vaccinated (3T3-OVA and 3T3-OVA-gp96) mice were analyzed for their CD103 expression. Gp96-Ig immunization induced dramatic increase of CD11c^{high} MHC class II high CD103⁺ cells compared to control mice (P<0.001) (FIGS. 14A and 14B). To investigate whether peritoneal CD103⁺ cells were required for priming and induction of gut-homing receptors on OT-I cells, carboxyfluorescein diacetate succinimidyl ester (CFSE)-labeled OT-I cells were co-cultured with sorted, Ova peptide (pOva; 2 n m)-pulsed CD103⁺ and CD103⁻ DCs from PEC of gp96 vaccinated or control mice (PBS). Expression of CCR9 on responding T cells was assessed 5 days later (FIGS. 14C and 14D). Both subsets CD103⁺ and CD103⁻ DCs from vaccinated and control animals induced proliferation of OT-I cells, but only CD103⁺ DCs induced CCR9 on responding OT-I cells (FIGS. 14C and 14D).

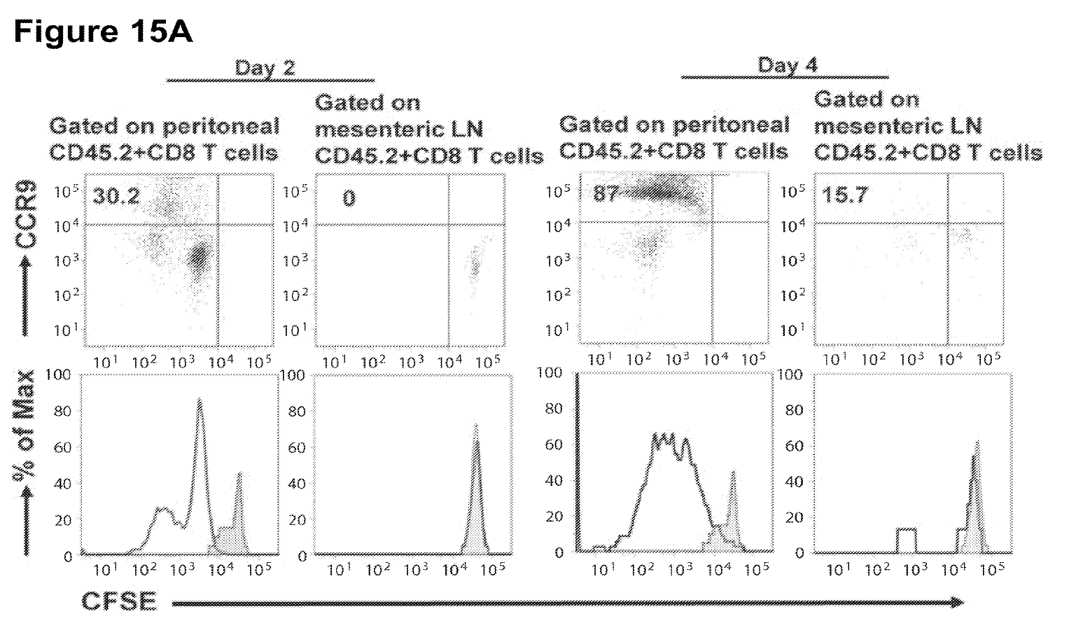

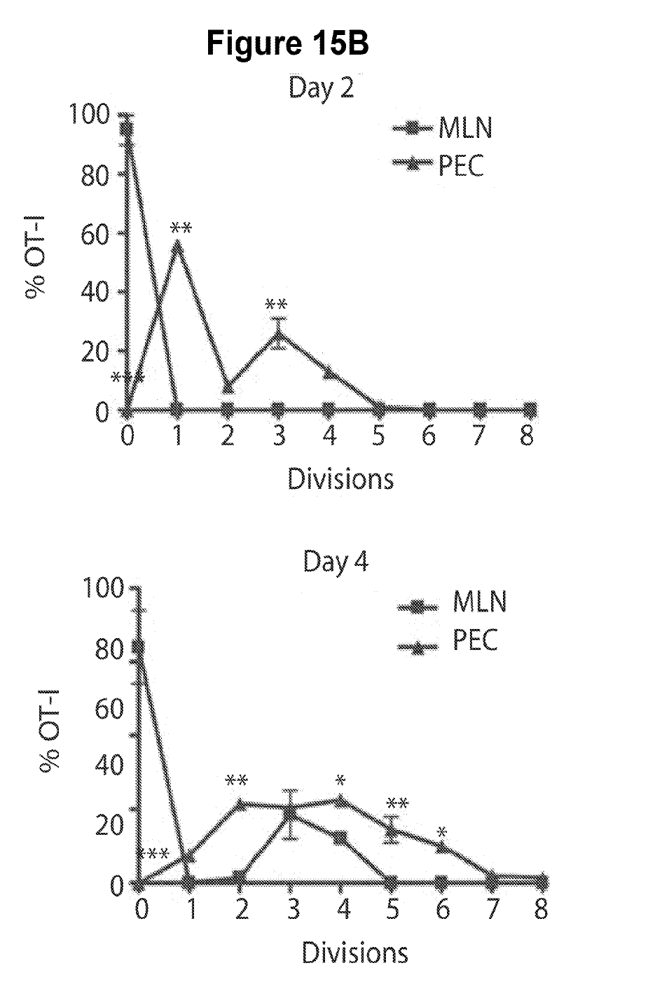

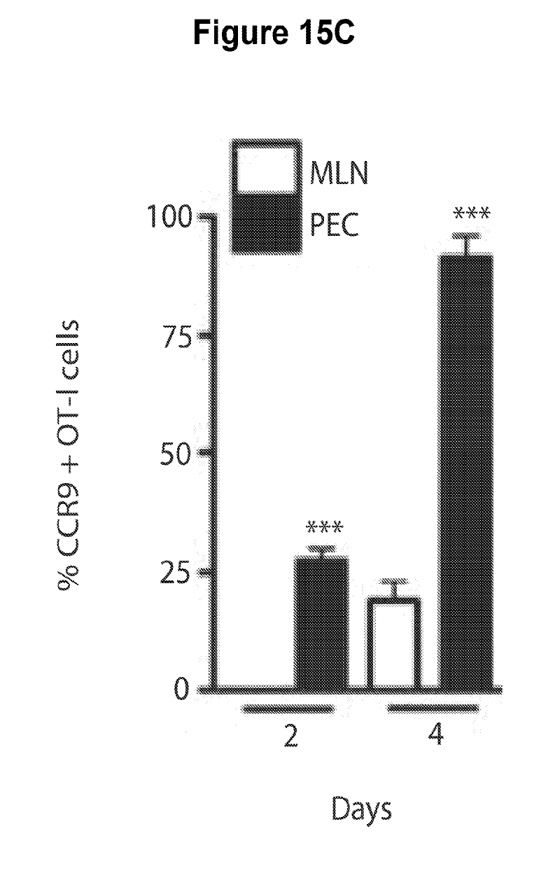

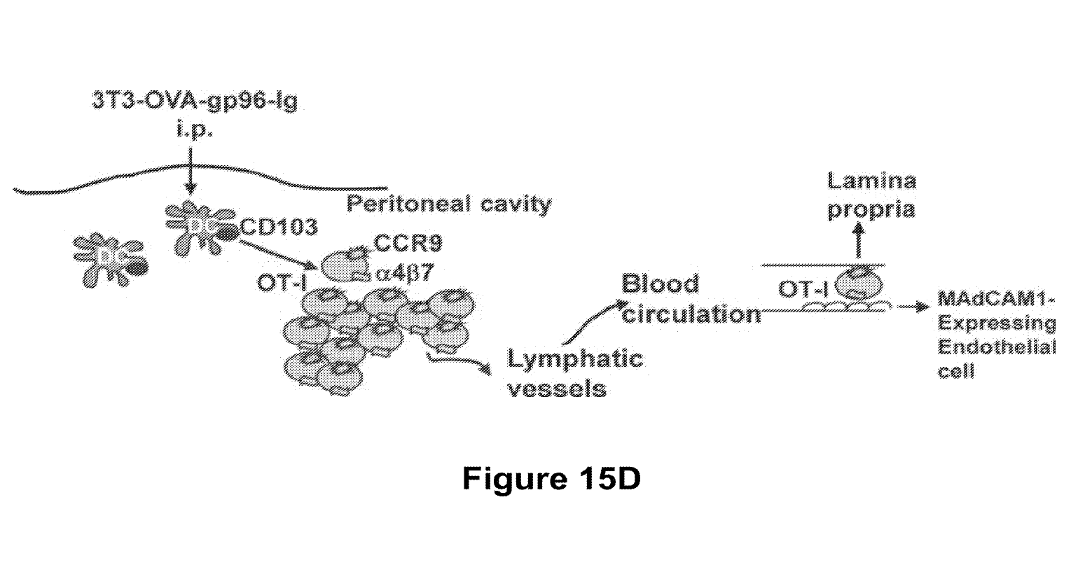

CD103⁺ DCs isolated from gp96 vaccinated mice induced comparable levels of CCR9 compared with CD103⁺ DCs from control animals. LTα (lymphotoxin-α)-deficient mice showed normal OT-I expansion in the PEC when compared with wild-type mice. This finding indicated that LNs are not essential for gp96-mediated peptide cross-priming and that local cross-priming takes place at the site of gp96 release. To determine whether priming for generation of gut-tropic T cells is occurring in the peritoneum as opposed to the intestinal LN, CFSE-labeled CD45.2⁺ OT-I cells were adoptively transferred into CD45.1⁺ wild-type recipient mice. Recipient mice were vaccinated with 3T3-OVA-gp96-Ig, and CCR9 expression on responding OT-I cells in PEC and mesenteric LNs was determined 2 and 4 days later (FIGS. 15A-15D). CD8 cells present in the PEC by day 2 showed significant increase in proliferation and CCR9 upregulation, whereas at the same time CD8 cells in mesenteric LNs did not proliferate and remained CCR9 negative (FIGS. 15A-15C). By day 4, gp96-dependent proliferation by CD8 cells in the PC was very pronounced and significantly higher than in the LNs (FIGS. 15A and 15B). Together, these results show remarkable properties of secreted gp96 to induce local, peritoneal activation/proliferation and migration of OT-I cells to the intestinal LP (FIG. 15C).

The IP Route of Immunization is Most Effective for Generating Mucosal and Systemic Immunity.

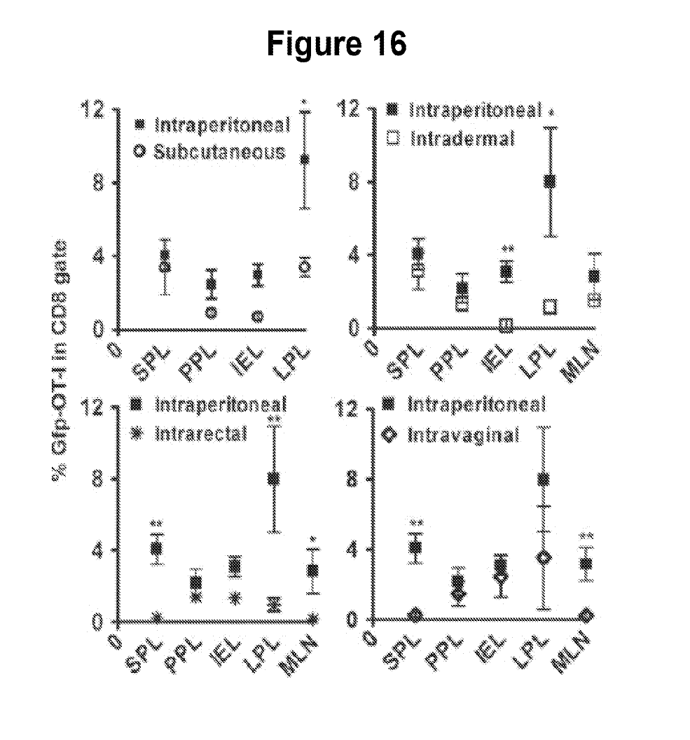

The physiological microenvironment of inductive sites with its characteristic resident populations, including macrophages, DCs, epithelial cells, B and T cells imprints the ensuing lymphocyte immune responses. Effector T cells generated in different lymphoid organs show distinct tissue tropisms, a feature that appears to be regulated by an organ-specific induction of adhesion molecules and chemokine receptors during T-cell priming. DCs are known to be the ‘key’ cells for imprinting of tissue-tropic T effector cells. Immunization with secreted gp96-Ig in the peritoneal environment seems to generate preferentially gut-tropic effector cells. To compare different routes of immunization by gp96-Ig, several vaccine priming sites were examined (FIG. 16). The IP route was twice as effective as the subcutaneous route for generating

mucosal and systemic immunity (LPL and IEL) while generating equal systemic immunity (SPL). Intradermal immunization with gp96-Ig generated the same degree of splenic and LN OT-I but fewer LPL OT-I than IP immunization. Interestingly, intradermal immunization did not induce migration of OT-I to the intraepithelial compartment. In contrast to IP immunization, intrarectal and intravaginal immunization by instillation of gp96-Ig-secreting cells induced only mucosal and systemic immunity; a systemic response in the SPL was undetectable. This finding indicates that the generation of immune responses in certain locations can be controlled by choosing appropriate vaccination routes.

Discussion

As gatekeepers of the immune response, DCs serve as the prime integrators of two pieces of environmental information—evidence for danger, e.g., inflammatory cytokines or microbial PAMP, and the antigenic signature of the pathogen. Although these are traditionally thought as two independent signals, extracellular gp96 packages both signals into a single complex targeting DCs. Gp96 itself activates and matures DCs and natural killer cells whereas the chaperoned peptide is cross-presented through MHC class I to CD8 T cells. This dual function makes extracellular gp96-peptide complexes extraordinarily efficient in priming CTL responses.

The data herein show that the gp96-Ig-peptide complexes fulfill the same role in mucosal and systemic CD8 CTL activation, clonal expansion, and trafficking to mucosal and systemic membranes. The gut mucosa is frequently exposed to pathogenic bacteria and viruses. In many epidemic diseases including typhoid fever, cholera, poliomyelitis and, most recently, HIV, the mucosa acts as the main port of entry. Secretory IgA and adaptive CD8⁺ IEL and LPL are clearly in a strategic location as early defenders against pathogenic attack to prevent invasion by pathogens or their spreading after initial infection. As to viral pathogens, a preventive vaccine must be able to generate mucosal and systemic immunity that neutralizes the virus or destroys infected cells before viral replication and spreading. Although virus neutralization is primarily achieved by antibody, killing of infected cells is the task primarily of adaptive CD8+ CTL. In particular intraepithelial, adaptive CD8⁺ IELs are in a frontline position to eradicate infected cells.

The cell-secreted gp96-Ig vaccines provide powerful stimuli for the expansion and differentiation of antigen-specific CD8 CTL systemically and were effective as protective and therapeutic tumor vaccines. Expanding the analysis to mucosal and systemic compartments, the secreted, gp96-Ig-based, cellular vaccines also induced antigen-specific CD8 CTL in the intraepithelial and LP compartments of the gut mucosa and therefore will have important uses for the development of mucosal and systemic vaccines.

The adoptive transfer system using gfp-marked TCR transgenic CD8T cells (OT-I) was used to quantify antigen-specific mucosal and systemic memory CD8T cells present in relevant anatomic sites and determine their phenotype and functional properties. These data evidence that vaccines that are able to induce high levels of adaptive mucosal and systemic CD8 effector cells may provide long-lasting immunity against infection with the specific pathogen. Vaccine induced CD8⁺ CTLs present at mucosal and systemic sites of infection delayed mucosal and systemic CD4 depletion by simian/human immunodeficiency virus whereas systemic presence of specific CD8 CTL had little effect on mucosal and systemic CD4 depletion.

The use of viruses and bacteria or of viral vectors or attenuated viruses for induction and analysis of the immune response relies, to a large extent, on the ability of viral or bacterial components to activate the immune system, e.g., through pattern recognition receptors. In the modality of gp96-Ig immunization described here, cells express the relevant antigen (OVA), which is used as reporter antigen together with TCR transgenic reporter cells. The same antigen-containing cells not secreting gp96-Ig are used as controls, restricting the observed immune response to the presence or absence of secreted gp96-Ig chaperoning the relevant peptides. Importantly, the use of allogeneic or syngeneic cells containing the antigen and secreting gp96-Ig elicited essentially the identical, antigen-specific immune response. This finding evidences that gp9-Ig-mediated immune activation is independent and possibly dominant over other immune responses, including allogeneic stimulation (NIH-3T3) or immune evasion by tumors (e.g., EG7—lymphoma).

Because the only difference between control and immune activation is the secretion of gp96-Ig, the observed immune response is entirely attributable to the adjuvant and cross-priming effect of gp96-peptide complexes. The IgG1-Fc tag used to replace the KDEL sequence in our study had no effect on the ensuing immune response. The use of cells secreting gp96-Ig described here, rather than the use of purified soluble gp96, had major effects on the potency of the immune response. Cell-secreted gp96-Ig is 10-20 times more potent than an equivalent amount of soluble gp96-Ig. This finding was interpreted to indicate that the immune system perceives continuous secretion and antigenic stimulation by gp96-Ig at the injection site as being equivalent to virus or bacterial replication, resulting in a vigorous immune responses. The data reported above are in support of this hypothesis and indicate that gp96-Ig-mediated mucosal and systemic (and systemic) immunity is quite comparable to the immune response induced by infectious agents.

Accordingly, it was shown herein, inter alia, that the IP immunization system, which relies on secreted heat shock fusion protein gp96-Ig, induced strong mucosal and systemic CD8-CTL responses in addition to systemic CTL responses. A major effect of gp96-Ig immunization was the rapid migration of OT-I into the LPL and intraepithelial compartments (IEL). Immigrating cells were clearly identifiable by expression of high levels of CD44, CD103, CCR9, and α4β7 and downregulation of CD62L and CCR7. The data also indicate that gp96-Ig-induced mucosal and systemic phenotypes had the general feature of gut memory. Furthermore, very high expression of the cytolytic mediator (granzyme B) was found in IEL OT-I and LPL OT-I (FIG. 12). The properties of gp96-Ig-induced mucosal and systemic CD8 CTL thus corresponded closely to those observed previously following lymphocytic choriomeningitis virus and vesicular stomatitis virus infection, including high granzyme B expression and low IL-2 and CD62L expression.

Although non-viral and nonbacterial in nature, gp96-mediated mucosal and systemic and systemic CD8 CTL responses bear the hallmarks of memory responses characteristically seen after viral or bacterial infections. This was attributed to the adjuvanticity of gp96, which is specifically directed toward cross-priming cellular, cytotoxic CD8-CTL responses. In addition the continuous secretion of gp96-Ig from the injected cells for up to 5 days, may, resemble viral replication and contribute to the cytotoxic response.

It is well appreciated that the site of immunization directs the imprinting of the ensuing T-cell response and controls the expression of trafficking molecules. The data herein, indicate that the best route of vaccination for induction of both systemic and mucosal and systemic CD8-CTL immunity is the IP route in mice. Similar data was also seen in nonhuman primates.

Gp96-Ig immunization increased the frequency of CD11c^{high} MHC class II^{high} CD103⁺ cells in PEC (FIG. 14A). Phenotypic analysis of CD11c^{high} MHC class II^{high} CD103⁺ cells revealed that these cells are CD8 negative, express CD11b, CD40, CD80, CCR7, B220, and low levels of CD86 and Gr-1. CD103⁺ CD11b⁺ and CD103⁺ CD8⁻ DC populations are more prominent in MLN and colonic LP. CD103⁺ DCs are the main migratory subtype with dominant cross-presenting ability, induction of CD103⁺ DCs by gp96 represents an ideal vaccination strategy for priming effective immunity. Whether the priming after IP administration of gp96 occurs in the milky spots of the greater omentum remains to be determined.

Gp96-Ig-induced gut-migrating OT-I cells represent type a IEL, which express high amounts of granzyme B, but at the same time after in vitro peptide stimulation produce less IFN-γ and IL-2 than splenic OT-I (FIGS. 13A-13D). Also, IEL OT-I did not produce IL-17, a cytokine that has been associated with autoimmune diseases 44 and virus-induced wasting disease in mice with CD8 T cells that lack both T-bet and Eomes and over-express IL-17. Without wishing to be bound by theory, antigen-specific IELs in the intestine respond to antigen stimulation with strong cytolytic activity and diminished cytokine secretion to prevent the development of intestinal immunopathology while eliminating infected cells.

In summary, gp96-Ig-induced antigen-specific CD8T cells have the ability to migrate to mucosal and systemic surfaces and provide immediate and enhanced protection at the most likely entry site of invading pathogens. Cell-secreted gp96-Ig immunization uses an internal danger signal, gp96, whose release by necrotic cell death sets off a cellular immune response directed at antigenic, chaperoned peptides. Use of this patho-immunological mechanism seems ideally suited for vaccine purposes, including stimulation of cellular mucosal and systemic immunity.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the disclosure will allow the reader to quickly ascertain the nature of the technical disclosure. It is submitted with the understanding that it will not be used to interpret or limit the scope or meaning of the following claims.

1-41. (canceled)

42. A method of inducing an antigen-specific immune response against a virus in a subject, the method comprising the step of administering to the subject a vaccine composition comprising a host cell engineered to co-express at least three different antigens from a primate lentivirus and a heat shock protein modified to be secreted from the host cell, wherein the antigens are selected from the group consisting of: at least a portion of Gag, at least a portion of Tat, at least a portion of Rev, a least a portion of Nef, and at least a portion of gp160.

43. The method of claim 42 wherein the host cell is a human cell.

44. The method of claim 42, wherein the modified heat shock protein is gp96 lacking a functional endoplasmic reticulum retention sequence.

45. The method of claim 42, wherein the host cell has been irradiated.

46. The method of claim 42, wherein the step of administering the vaccine results in expansion of T cells specific for the antigens in the subject's peripheral blood, and the method further comprises the step of analyzing the subject's peripheral blood for expansion of T cells specific for the antigens.

47. The method of claim 42, wherein the step of administering the vaccine results in expansion of T cells specific for the antigens in the subject's mucosa, and the method further comprises the step of analyzing the subject's mucosa for expansion of T cells specific for the antigens.
